SEQUENCE LISTING (52 sequences)

SEQ ID NO: 1 - protein, 124 aa, Artificial Sequence
  HER3-MF3178
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 2 - protein, 122 aa, Artificial Sequence
  HER2-MF1849
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGDYGSYSSYAFDYWGQGTLVTVSS

SEQ ID NO: 3 - protein, 120 aa, Artificial Sequence
  EGFR-MF3755
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 4 - protein, 125 aa, Artificial Sequence
  EGFR-MF4280
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 5 - protein, 119 aa, Artificial Sequence
  EGFR-MF4003
QVQLVQSGSELKKPGASVKVSCKASGYTFPSFAMNWLRQAPGQGLEWMGWITTNTGDPTYAQGFSGRFVFSLDTSVSTAYLQISSLKAEDTAVYYCARVYNWIRGFDYWGQGTLVTVSS

SEQ ID NO: 6 - protein, 122 aa, Artificial Sequence
  HER2-MF2032
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYYRRTARAGFDYWGQGTLVTVSS

SEQ ID NO: 7 - protein, 6 aa, Artificial Sequence
  VL CDR1
QSISSY

SEQ ID NO: 8 - protein, 7 aa, Artificial Sequence
  VL CDR2
AASSLQS

SEQ ID NO: 9 - protein, 7 aa, Artificial Sequence
  VL CDR3
QQSYSTP

SEQ ID NO: 10 - protein, 95 aa, Artificial Sequence
  IgVk1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 11 - protein, 107 aa, Artificial Sequence
  Common Light Chain IgKV1*39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 12 - protein, 108 aa, Artificial Sequence
  Common Light Chain IgKV1*39/jk5
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 13 - protein, 113 aa, Artificial Sequence
  IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 14 - protein, 110 aa, Artificial Sequence
  IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, Artificial Sequence
  IgG1 CH3 KK
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 107 aa, Artificial Sequence
  IgG1 CH3 DE
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 121 aa, Artificial Sequence
  MF1847 HER2
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGWWHPLLSGFDYWGQGTLVTVSS

SEQ ID NO: 18 - protein, 121 aa, Artificial Sequence
  MF1871 HER2
EVQLVQSGAEVKKPGESLKISCKGSGYSFTSYWIGWVRQMPGKGLEWMGIIYPGDSDTRYSPSFQGQVTISADKSISTAYLQWSSLKASDTAVYYCARGRYDLWWYGFDYWGQGTLVTVSS

SEQ ID NO: 19 - protein, 119 aa, Artificial Sequence
  MF4000 EGFR
EVQLVESGGGLVKPGGSLRLSCAASGFTFNSYSIHWVRQAPGEGLEWVSFISSSSEYIYYADSVKGRFTISRDNAKNSLYLQMNSLRADDTAVYYCARGVGAPHAFDIWGQGTMVTVSS

SEQ ID NO: 20 - protein, 125 aa, Artificial Sequence
  MF4290 EGFR
QMQLVQSGAEVKKPGASVKVSCKISGYTLTELSMHWVRQAPGKGPEWMGGFDPEYGETFFAQQFQGRVTMTEDTSTDTAYMELSSLRSEDTAIYYCATEGYYQTTSYYYNLFDYWGQGTLVTVSS

SEQ ID NO: 21 - protein, 126 aa, Artificial Sequence
  MF4016 EGFR
EVQLVESGGDLVQPGGSLRLSCAASGFTFSSYAMNWVRQAPGKGLEWVSAISGSGSSTYYADSVKGRFTISRDNSKNTLYLQMNSLRAEDTAVYYCAKEPNYYGSGSPHYFYMDVWGKGTTVTVSS

SEQ ID NO: 22 - protein, 127 aa, Artificial Sequence
  MF2703 HER3
EVQLVESGGGLVQPGGSLRLSCAASGFTFSSYAMSWVRQAPGKGLEWVSTFSGSDGNTYYADSVKGRFTISRDNSKNMLNLYMDSLRAEDTAVYYCAKDSDYSSGWYGFPTDYFDYWGQGTLVTVSS

SEQ ID NO: 23 - protein, 125 aa, Artificial Sequence
  MF2708
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSS

SEQ ID NO: 24 - protein, 127 aa, Artificial Sequence
  MF1337 Tetanus toxoid
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 25 - protein, 30 aa, Artificial Sequence
  MF3755 EGFR FR1
QVQLVQSGSELKKPGASVKISCKASGYDFT

SEQ ID NO: 26 - protein, 5 aa, Artificial Sequence
  MF3755 EGFR CDR1
NYAMN

SEQ ID NO: 27 - protein, 14 aa, Artificial Sequence
  MF3755 EGFR FR2
WVRQAPGHGLEWMG

SEQ ID NO: 28 - protein, 17 aa, Artificial Sequence
  MF3755 EGFR CDR2
WINANTGDPTYAQGFTG

SEQ ID NO: 29 - protein, 32 aa, Artificial Sequence
  MF3755 EGFR FR3
RFVFSLDTSVSTAYLQISSLKAEDSAVYYCTR

SEQ ID NO: 30 - protein, 11 aa, Artificial Sequence
  MF3755 EGFR CDR3
ERFLEWLHFDY

SEQ ID NO: 31 - protein, 11 aa, Artificial Sequence
  MF3755 EGFR FR4
WGQGTLVTVSS

SEQ ID NO: 32 - protein, 30 aa, Artificial Sequence
  MF3178 HER3 FR1
QVQLVQSGAEVKKPGASVKVSCKASGYTFT

SEQ ID NO: 33 - protein, 5 aa, Artificial Sequence
  MF3178 HER3 CDR1
GYYMH

SEQ ID NO: 34 - protein, 14 aa, Artificial Sequence
  MF3178 HER3 FR2
WVRQAPGQGLEWMG

SEQ ID NO: 35 - protein, 17 aa, Artificial Sequence
  MF3178 HER3 CDR2
WINPNSGGTNYAQKFQG

SEQ ID NO: 36 - protein, 32 aa, Artificial Sequence
  MF3178 HER3 FR3
RVTMTRDTSISTAYMELSRLRSDDTAVYYCAR

SEQ ID NO: 37 - protein, 15 aa, Artificial Sequence
  MF3178 HER3 CDR3
DHGSRHFWSYWGFDY

SEQ ID NO: 38 - protein, 30 aa, Artificial Sequence
  MF1849 HER2 FR1
QVQLVESGGGVVQPGRSLRLSCAASGFTFS

SEQ ID NO: 39 - protein, 5 aa, Artificial Sequence
  MF1849 HER2 CDR1
SYGMH

SEQ ID NO: 40 - protein, 14 aa, Artificial Sequence
  MF1849 HER2 FR2
WVRQAPGKGLEWVA

SEQ ID NO: 41 - protein, 17 aa, Artificial Sequence
  MF1849 HER2 CDR2
VISYDGSNKYYADSVKG

SEQ ID NO: 42 - protein, 32 aa, Artificial Sequence
  MF1849 HER2 FR3
RFTISRDNSKNTLYLQMNSLRAEDTAVYYCAK

SEQ ID NO: 43 - protein, 13 aa, Artificial Sequence
  MF1849 HER2 CDR3
GDYGSYSSYAFDY

SEQ ID NO: 44 - protein, 13 aa, Artificial Sequence
  MF2032 HER2 CDR3
DYYRRTARAGFDY

SEQ ID NO: 45 - protein, 30 aa, Artificial Sequence
  MF1337 Tetanus toxoid FR1
EVQLVETGAEVKKPGASVKVSCKASDYIFT

SEQ ID NO: 46 - protein, 5 aa, Artificial Sequence
  MF1337 Tetanus toxoid CDR1
KYDIN

SEQ ID NO: 47 - protein, 14 aa, Artificial Sequence
  MF1337 Tetanus toxoid FR2
WVRQAPGQGLEWMG

SEQ ID NO: 48 - protein, 17 aa, Artificial Sequence
  MF1337 Tetanus toxoid CDR2
WMSANTGNTGYAQKFQG

SEQ ID NO: 49 - protein, 32 aa, Artificial Sequence
  MF1337 Tetanus toxoid FR3
RVTMTRDTSINTAYMELSSLTSGDTAVYFCAR

SEQ ID NO: 50 - protein, 18 aa, Artificial Sequence
  MF1337 Tetanus toxoid CDR3
SSLFKTETAPYYHFALDV

SEQ ID NO: 51 - protein, 11 aa, Artificial Sequence
  MF1337 Tetanus toxoid FR4
WGQGTTVTVSS

SEQ ID NO: 52 - protein, 6 aa, Artificial Sequence
  VL CDR2
AASLQS

(19) United States
(12) Patent Application Publication
(10) Pub. No.: US 2022/0127376 A1
(43) Pub. Date: Apr. 28, 2022

(54) COMBINATIONS OF BINDING MOIETIES THAT BIND EGFR, HER2 AND HER3

(71) Applicant: Merus N.V., Utrecht (NL)

(72) Inventors: Cecilia Anna Wilhelmina GEUIJEN, Utrecht (NL); Tristan Louis Jean GALLENNE, Utrecht (NL); Mark THROSBY, Utrecht (NL); Cornelis Adriaan DE KRUIF, Utrecht (NL)

(73) Assignee: Merus N.V., Utrecht (NL)

(21) Appl. No.: 17/431,044

(22) PCT Filed: Feb. 13, 2020
(86) PCT No.: PCT/NL2020/050081

§ 371 Date: Aug. 13, 2021

(30) Foreign Application Priority Data
Feb. 14, 2019 (EP) 19157302.1
Jun. 5, 2019 (EP) 19178564.1

(51) Int. Cl. C07K 16/32; C07K 16/28; A61P 35/00

(52) U.S. Cl. CPC C07K 16/32; A61K 2039/507; A61P 35/00; C07K 16/2863

(57) ABSTRACT

The invention provides a composition comprising two or more binding moieties wherein each of each of said binding moieties comprises a variable domain that binds to an extracellular part of EGFR; and wherein a first of said binding moieties comprises a variable domain that binds to an extracellular part of HER2 and a second of said binding moieties comprises a variable domain that binds to an extracellular part of HER3. The invention also relates to means and method for producing compositions and for the treatment of subjects with the compositions.

The invention relates to the field of binding moieties such as antibodies, in particular to the field of therapeutic binding moieties. The binding moieties can be used in the treatment of humans. More in particular the invention relates to a composition comprising two or more multispecific binding moieties, preferably multispecific antibodies. The binding moieties bind EGFR, HER2 and HER3. A single host cell can produce the multiple binding moieties.

The epidermal growth factor (EGF) receptor (EGFR) is the prototype cell-surface receptor for members of the epidermal growth factor family (EGF-family) of extracellular protein ligands. The family presently has four closely related receptor tyrosine kinases: EGFR, HER2 (ErbB-2/c-neu), HER3 (ErbB-3) and HER4 (ErbB-4).

EGFR exists on the cell surface and is activated by binding of its specific ligands, including epidermal growth factor and transforming growth factor α (TGFα). Upon activation by its growth factor ligands, the receptor undergoes a transition from an inactive mostly monomeric form to an active homo-dimer. In addition to forming homo-dimers after ligand binding, EGFR may pair with another member of the ErbB receptor family, such as HER2, to create an activated hetero-dimer. There is also evidence to suggest that dimers form in the absence of ligand-binding and clusters of activated EGFRs form after ligand binding.

EGFR dimerization stimulates its intrinsic intracellular protein-tyrosine kinase (PTK) activity. This activity induces several signal transduction cascades that lead to cell proliferation and differentiation. The kinase domain of EGFR can cross-phosphorylate tyrosine residues of other receptors it is complexed with, and can itself be activated in that manner.

Mutations and overexpression involving EGFR have been identified in several types of cancer, and it is the target of an expanding class of anticancer therapies. These include EGFR targeted small molecules as gefitinib and erlotinib for lung cancer, and antibodies as cetuximab and panitumumab for colon cancer and head and neck cancer.

Although there is some success with the EGFR targeted therapies, most are associated with the development of treatment resistance over time. One of the ways in which EGFR positive tumors can escape the targeted therapy is by signaling through another receptor dimer. For instance, increased signaling by EGFR/HER3 dimers due to increased HER3 expression or heregulin expression is associated with EGFR targeted drug resistance in, for example, lung cancers and head and neck cancers. Apart from the induction of treatment resistance, some side effects of EGFR-targeting antibodies have been observed. One example is the development of a skin rash, associated with EGFR inhibition or anti-EGFR biologic treatment. When extreme, such rashes can lead to a reduction in treatment cycles and/or premature termination of treatment.

Various modes of activation of signaling of the EGF receptor family have been identified. Among these are ligand dependent and ligand independent activation of signaling. Over-expressed HER2 is able to generate oncogenic signaling through the HER2/HER3 heterodimer even in the absence of the HER3 ligand (Junttila, Akita et al. 2009). HER2 activity can be inhibited by HER2 specific antibodies. Such HER2 specific antibodies are for instance used in the treatment of HER2 positive (HER2+) tumors. A problem with such treatments is that often tumors escape the HER2 specific treatment and continue to grow even in the presence of the inhibiting antibody. It has been observed that. HER2 positive tumors, such as breast, ovarian, cervical and gastric tumors can escape treatment by the selective outgrowth of a subpopulation of tumor calls that exhibit upregulated HER3 expression (Ocana, Vera-Badillo et al. 2013) and/or HER3 ligand expression (Wilson, Fridlyand et al. 2012). Also activating mutations in the HER3 receptor have been identified.

Thus, in spite of promising results with antibody treatments that specifically target EGF receptor family members, it has been observed that not all tumors respond or respond sufficiently. The present invention provides combinations of binding moieties and methods for producing them that target various members of the EGF receptor family. Combinations of the invention show good efficacy. The combinations can be produced in a cost effective and efficient way.

### SUMMARY OF THE INVENTION

The invention provides a composition comprising two or more binding moieties,

wherein each binding moiety comprises a variable domain that binds to an extracellular part of EGFR; and

wherein a first of said binding moieties comprises a variable domain that binds to an extracellular part of HER2 and a second of said binding moieties comprises a variable domain that binds to an extracellular part of HER3.

Preferably, at least one of the two or more binding moieties is an antibody. In a preferred embodiment, at least two of the two or more binding moieties are antibodies. The antibodies are preferably multispecific antibodies, preferably bispecific antibodies. Preferably at least one, and more preferably at least two of the antibodies are IgG antibodies. In a preferred embodiment of the invention, the composition comprising two bispecific antibodies.

A multispecific antibody as described herein preferably comprises heavy chains with a CH3 heterodimerization domain. In one embodiment the CH3 heterodimerization domain of the first and/or the second multispecific antibody is engineered to facilitate heterodimerization of the heavy chain of the EGFR variable domain with the respective heavy chains of the HER2 and HER3 variable domains.

The invention also provides a composition as described herein for use in the treatment of cancer. In embodiments the cancer is a solid epithelial cancer. Preferably the composition is used for a cancer that expresses EGFR, HER2 and/or HER3. The composition is used for preferably for pancreatic cancer, colorectal cancer, head & neck cancer, epithelial ovarian cancer, epithelial fallopian tube cancer, epithelial peritoneal cancer, bladder cancer, or prostate cancer. In embodiments the cancer treated by use of the composition is advanced cancer. The composition is used for preferably for metastatic cancer. The composition is used for preferably for metastatic pancreatic cancer, metastatic colorectal cancer, metastatic head & neck cancer, metastatic epithelial ovarian cancer, metastatic epithelial fallopian tube cancer, metastatic epithelial peritoneal cancer, metastatic bladder cancer, or metastatic prostate cancer. In embodiments, the composition is used for preferably for the cancer that is gastric cancer, lung cancer, breast cancer or esophagus cancer. Preferably, the composition is used for metastatic gastric cancer, metastatic lung cancer, metastatic breast cancer or metastatic esophagus cancer.

The invention further provides a product containing two or more binding moieties that each comprise a variable domain that binds to an extracellular part of EGFR; wherein a first of said binding moieties comprises a variable domain that binds to an extracellular part of HER2 and a second of said binding moieties comprises a variable domain that binds to an extracellular part of HER3 as a combined preparation for simultaneous, separate or sequential use in treating cancer.

The invention further provides a method for producing a composition according to the invention, which method comprises:

providing a cell comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with said common light chain forms a variable domain that binds to an extracellular part of HER2;
a nucleic acid that encodes a polypeptide comprising a heavy chain that together with said common light chain forms a variable domain that binds to an extracellular part of HER3; and
a nucleic acid that encodes a polypeptide comprising said common light chain;
   wherein two or more of said nucleic acids may be physically linked or not and wherein each of said nucleic acids further comprises an expression control sequence to allow expression of the encoded heavy and light chains in said cell and wherein the method further comprises culturing said cell to allow expression of said heavy and light chains and, optionally collecting said two or more binding moieties.

Further provided is a cell comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with said common light chain forms a variable domain that binds to an extracellular part of HER2;
a nucleic acid that encodes a polypeptide comprising a heavy chain that together with said common light chain forms a variable domain that binds to an extracellular part of HER3; and
a nucleic acid that encodes a polypeptide comprising said common light chain;
   wherein two or more of said nucleic acids may be physically linked or not and wherein each of said nucleic acids further comprises an expression control sequence to allow expression of the encoded heavy and light chains in said cell.

In a further aspect the invention provides a container comprising nucleic acid comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with a common light chain forms a variable domain that binds to an extracellular part of HER2;
a nucleic acid that encodes a polypeptide comprising a heavy chain that together with a common light chain forms a variable domain that binds to an extracellular part of HER3; and
a nucleic acid that encodes a polypeptide comprising said common light chain;

wherein, optionally, two or more of said nucleic acids may be physically linked or not and wherein each of said nucleic further comprises an expression control sequence to allow expression of the encoded heavy and light chains in a cell.

The invention further provides a composition comprising a binding moiety that specifically binds an extracellular part of EGFR and an extracellular part of HER2.

The invention also provides composition comprising a binding moiety that specifically binds an extracellular part of EGFR and an extracellular part of HER3.

The binding moiety is preferably an antibody, preferably an IgG antibody, more preferably a multi-specific antibody.

### DETAILED DESCRIPTION OF THE INVENTION

The term EGFR as used herein refers to the protein that in humans is encoded by the epidermal growth factor receptor gene (EGFR). The protein is known under a number of aliases among which: Erb-82 Receptor Tyrosine Kinase 1; Proto-Oncogene C-ErbB-1; ERBB1; and HER1. A database accession number for the human EGFR protein and the gene encoding it is (GenBank NM_005228.3). The accession number is primarily given to provide a further method of identification of EGFR protein as a target, the actual sequence of the EGFR protein bound by an antibody may vary, for instance because of a mutation in the encoding gene such as those occurring in some cancers or the like. Where reference herein is made to EGFR, the reference refers to human EGFR unless otherwise stated. The EGFR variable domain may, due to sequence and tertiary structure similarity between human and other mammalian orthologues of EGFR, also bind such an orthologue but not necessarily so. The variable domain that binds EGFR, may bind EGFR and a variety of variants thereof such as those expressed on some EGFR positive tumors.

The EGFR binding variable domain of an antibody or binding moiety of the invention preferably binds domain I or domain III of EGFR. The structure of the EGFR protein has been described among others in Ferguson (2008: Annu Rev Biophys. 2008:37:353-373. doi: 10.1146/annurev.biophys.37.0:32807.125829). The domains of human EGFR are described in FIG. 1 of the above mentioned Ferguson reference. The EGFR binding variable domain of embodiments of the inventions disclosed herein preferably binds domain III of EGFR. The antibody preferably inhibits EGF induced proliferation of BxPC-3 (ATCC CRL-1687) or BxPC-3-luc2 cells (Perkin Elmer 125058).

The term HER2 as used herein refers to the protein that in humans is encoded by the ERBB-2 gene. Alternative names for the gene or protein include CD340; ErbB-2: HER-2/neu; MLN 19; NEU; NGL: TKR1. The ERBB-2 gene is frequently called HER2 (from human epidermal growth factor receptor 2). Where reference is made herein to HER2, the reference refers to human HER2. An antibody comprising a variable domain that binds HER2, binds human HER2. The HER2 variable domain may, due to sequence and tertiary structure similarity between human and other mammalian orthologues of HER2, also bind such an orthologue but not necessarily so. Database accession numbers for the human HER2 protein and the gene encoding it are (NP_001005862.1, NP_004439.2, NC_000017.10, NT_010783.15). The accession numbers are primarily given to provide a further method of identification of HER2 as a target, the actual sequence of the HER2 protein bound the antibody may vary, for instance because of a mutation in the encoding gene such as those occurring in some cancers or the like. The HER2 variable domain may bind HER2 and a variety of variants thereof, such as those expressed by some HER2 positive tumor cells.

The HER2 protein contains several domains (see for reference FIG. 1 of Landgraf, R Breast Cancer Res. 2007; 9(1): 202-). The extracellular domains are referred to as domains I-IV. A variable domain of embodiments of the inventions disclosed herein that binds HER2 preferably binds domain I or domain IV of HER2, preferably domain IV.

The term HER3 as used herein refers to the protein that in humans is encoded by the ERBB3 gene. Alternative names for the gene or protein are LCCS2; MDA-BF-1; c-ErbB-3; c-ErbB3; ErbB3-S; p180-ErbB3; p45-sErbB3; and p85-sErbB3. Where reference is made herein to HER3, the reference refers to human HER3. An antibody comprising a variable domain that binds HER3, binds human HER3. The HER3 variable domain may, due to sequence and tertiary structure similarity between human and other mammalian HER3 orthologues, also bind such an orthologue but not necessarily so. Database accession numbers for the human HER3 protein and the gene encoding it are (NP_001.005915.1; NP_001973.2. NC_000012.11, NT_02941.9.12). The accession numbers are primarily given to provide a further method of identification of HER3 as a target, the actual sequence of the HER3 protein bound by an antibody may vary, for instance because of a mutation in the encoding gene such as those occurring in some cancers or the like. The HER3 variable domain may bind HER3 and a variety of variants thereof, such as those expressed by some HER2 positive tumor cells.

The structure of HER3 is among others described in Cho et al (2002: Science 297, 1330-1333: DM: 10.1126/science.1074611). The human protein has four extracellular domains. The variable domain of embodiments of the inventions disclosed herein that binds HER3 preferably binds domain III of HER3. In a preferred embodiment the affinity (KD) of a variable domain for an HER3 positive cell is lower than or equal to 2.0 nM, more preferably lower than or equal to 1.5 nM, more preferably lower than or equal to 1.39 nM, more preferably lower than or equal to 0.99 nM. In a preferred embodiment, an antibody according to the invention preferably comprises a variable domain that binds at least one amino acid of domain III of HER3 selected from the group comprising of R426 and amino acid residues that are located within 11.2 Å from R426 in the native HER3 protein. In one preferred embodiment, the affinity (KD) of a variable domain for HER3 on SK-BR-3 cells is lower than or equal to 2.0 nM, more preferably lower than or equal to 1.5 nM, more preferably lower than or equal to 1.39 nM, preferably lower than or equal to 0.99 nM. In one embodiment, said affinity (KD) is within the range of 1.39-0.59 nM. In one preferred embodiment, the affinity (KD) a variable domain for HER3 on BT-474 cells is lower than or equal to 2.0 nM, more preferably lower than or equal to 1.5 nM, more preferably lower than or equal to 1.0 nM, more preferably lower than 0.5 nM, more preferably lower than or equal to 0.31 nM, more preferably lower than or equal to 0.23 nM. In one embodiment, said affinity (ND) is within the range of 0.31-0.15 nM. The above-mentioned affinities are preferably as measured using steady state cell affinity measurements, wherein cells are incubated at 4° C., using radioactively labeled antibody, where after cell-bound radioactivity is measured.

A variable domain that binds at least one amino acid of domain III of HER3 preferably binds an amino acid selected from the group comprising R426 and amino acid residues that are located within 11.2 Å from R426 in the native HER3 protein. Preferably, said amino acid residues that are located within 11.2 Å from 8426 in the native HER3 protein are selected from the group consisting of L423, Y424. N425, G427. G452, R453. Y455, E480, R481, L482, D483 and K485 (see for instance FIG. 9 and Table 1). The amino acid residue numbering is that of Protein Data Bank (PDB) ID #4P59. Antibodies binding this region of domain III of HER3 exhibit particularly good binding characteristics and they are capable of counteracting an activity of HER3 on HER3 positive cells. Variable domains with the HER3 binding characteristics are described in WO2015/130172 which is incorporated by reference herein. In one preferred embodiment, a bispecific antibody according to the invention is provided, wherein said antibody comprises a variable domain that binds at least 8426 of domain III of HER3. Preferably, said antibody comprises a variable domain that binds at least 8426 of domain III of HER3.

In some embodiment a composition comprising two or more antibodies wherein each of said antibodies comprises a variable domain that binds to an extracellular part of EGFR; and wherein a first of said antibodies comprises a variable domain that binds to an extracellular part of HER2 and a second of said antibodies comprises a variable domain that binds to an extracellular part of HER3. In a preferred embodiment the variable domains that bind to an extracellular part of EGFR of the first and second antibody have essentially the same amino acid sequence. In one embodiment said first and second antibody comprise a variable domain that binds domain I of EGFR, and said first antibody comprises a variable domain that binds domain 1 of HER2 and said second antibody comprises a variable domain that binds domain III of HER3. In another embodiment said first and second antibody comprise a variable domain that binds domain I of EGFR, and said first antibody comprises a variable domain that binds domain IV of HER2 and said second antibody comprises a variable domain that binds domain III of HER3. In a further embodiment said first and second antibody comprise a variable domain that binds domain III of EGFR, and said first antibody comprises a variable domain that binds domain I of HER2 and said second antibody comprises a variable domain that binds domain III of HER3. In a further embodiment said first and second antibody comprise a variable domain that binds domain III of EGFR, and said first antibody comprises a variable domain that binds domain IV of HER2 and said second antibody comprises a variable domain that binds domain III of HER3.

In some embodiments a binding moiety is a protein or an aptamer. A binding moiety as described herein typically has two or more binding specificities. The binding moiety preferably comprises two or more variable domains of antibodies. Variable domains can be provided in various ways. A number of antibody variable domain containing fragments are described in “Nelson 2010: MAbs. 2010 January-February; 2(1):77-83” and include the various FAB fragments, scFv fragments and so-called single domain antibodies such as VHH fragments. The various FAB fragments or single chain Fv fragments are presently well known. A single-domain antibody is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Single domain fragments were first made from camelid heavy chain antibodies. Similar single domain fragments can now be made artificially and be derived from other organisms. A variable domain preferably comprises a heavy chain variable region and a light chain variable region. Variable domains are sometimes also referred to as VH/VL combinations where VH stands for variable region of the heavy chain and VL for the variable region of the light chain.

The two or more fragments can be linked to create binding moieties with as many binding specificities. Linkage is typically done with a linking peptide comprising 2 or more amino acid residues. Linking moieties can also be part or all of a protein. For instance, human serum albumin is sometimes used. Binding moieties as described herein preferably comprise at least one variable domain with a heavy chain variable region of an MF, for example as described in FIG. 7 or FIG. 8, paired with a light chain variable region, for example a light chain variable region. In a preferred embodiment the binding moiety comprises two or more of such variable domains.

The binding moiety that binds EGFR and HER2 is a different binding moiety than the binding moiety that binds EGFR and HER3. In the event that at least one of the binding moieties is a multispecific antibody, at least one multispecific antibody may bind at least EGFR and HER2 or bind at least EGFR and HER3. In a preferred embodiment the binding moieties comprise bispecific antibodies wherein one bispecific antibody hinds EGFR and HER2 and another bispecific antibody binds EGFR and HER3.

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units—each with two heavy chains and two light chains. Antibodies for therapeutic use are preferably as close to natural antibodies of the subject to be treated as possible (for instance human antibodies for human subjects). An antibody according to the present invention is not limited to any particular format or method of producing it.

As an antibody typically recognizes an epitope of an antigen, and such an epitope may be present in other compounds as well, antibodies according to the present invention that “specifically recognize” an antigen, for example, EGFR. HER2 or HER3, may recognize other compounds as well, if such other compounds contain the same kind of epitope. Hence, the terms “specifically recognizes” or “specifically binds” or terms with the same connotation, with respect to an antigen and antibody interaction does not exclude binding of the antibodies to other compounds that contain the same or the same kind of epitope.

A “bispecific antibody” is an antibody as described herein wherein one variable domain of the antibody binds to a first antigen whereas a second variable domain of the antibody binds to a second antigen, wherein said first and second antigens are not identical. The term “bispecific antibody” also encompasses antibodies wherein one heavy chain variable region/light chain variable region (VH/VL) combination binds a first epitope on an antigen and a second VH/VL combination binds a second epitope. The second epitope can be a different epitope on the same antigen. The term further includes antibodies wherein a VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236. WO 2010/108127 and Schaefer et al (Cancer Cell 20, 472-486, October 2011). A bispecific antibody according to the present invention is not limited to any particular bispecific format or method of producing it.

A bispecific antibody is an example of a multispecific antibody. A tri- and further specific antibody can be made by adding a binding moiety such as a scFv fragment to one or more of the heavy chains. It is also possible to add one or more variable domains to a variable region of a normal or bispecific antibody. A cell that produces a common light chain and two different heavy chains that each can form a functional variable domain with the common light chain, produces among others a bispecific antibody with two different heavy and light chain combinations. Similarly, a cell that produces a common light chain and three or more different heavy chains can form several bispecific antibodies that together are capable of targeting three or more antigens. Presently it is possible to build on the standard format of antibodies (i.e. a constant part and two variable domains) and add further binding domains. As such multispecific antibodies can be made that have one or more single chain Fv with additional binding specificities attached to the constant or one or more of the variable domains of an antibody. It is also possible to produce heavy chains with two or more variable regions. The additional heavy chain regions can advantageously associate with different or common light chain variable regions. Reference is made to U.S. 62/650,467 for a description of such antibodies which is incorporated by reference herein.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Program Advance 10.5.2 software using the default settings, which employ a modified ClustalW algorithm (Thompson, J. U., Higgins, U. G., and Gibson T. J. (1994) Nuc. Acid Res. 22: 4673-4680), the swgapdnarnt score matrix, a gap opening penalty of 15 and a gap extension penalty of 6.66. Amino acid sequences are aligned with the AlignX application of the Vector NTI Program Advance 11.5.2 software using default settings, which employ a modified ClustalW algorithm (Thompson, J. D., Higgins. D. G., and Gibson T. J., 1994), the blosum62mt2 score matrix, a gap opening penalty of 10 and a gap extension penalty of 0.1.

The term ‘common light chain’ as used herein refers to the light chains such as those that can be used in a multispecific antibody. In bispecific antibodies the two light chains can be a common light chain (or the VL part thereof). The two light chains (or the VL part thereof) may be identical or have some amino acid sequence differences while the binding specificity of the full length antibody is not affected. The terms ‘common light chain’, ‘common VL’, ‘single light chain’, ‘single VL’, with or without the addition of the term ‘rearranged’ are all used herein interchangeably. “Common” refers to light chains that have the same sequence and also refers to functional equivalents of which the amino acid sequence is not identical. Many variants of said light chain exist wherein mutations (deletions, substitutions, insertions and/or additions) are present that do not influence the formation of functional binding regions. The light chain of the present invention can also be a light chain as specified herein, having from 0 to 10, preferably from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. In some embodiments multispecific antibodies with three or more variable domains have variable domains with different heavy chains and the same light chain or a light chain with some amino acid differences while the binding specificity of the full length multispecific antibody is not affected. Such a light chain is advantageously also a common light chain as described herein. In a preferred embodiment all of the variable domains of a multispecific antibody comprise a common light chain. A common light chain (variable region) for use in the multivalent antibodies of the invention can be a lambda light chain and this is therefore also provided in the context of the invention, however a kappa light chain is preferred. The common light chain of the invention may comprise a constant region of a kappa or a lambda light chain. It is preferably a constant region of a kappa light chain, preferably wherein said common light chain is a germline light chain, preferably a rearranged germline human kappa light chain comprising the IgVK1-39 gene segment, for example the rearranged germline human kappa light chain IgVK1-39*01/IGJκ1*01. Examples of common light chain amino acid sequences are indicated in FIG. 7 sequence 10, 11 or 12).

The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2. CH3, VH, and CL. VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen or epitope binding specificity of the IgG. Examples of IgG antibodies are IgG1, IgG2, IgG3 and IG4 antibodies. In some embodiments of the invention the IgG is an IgG1.

Preferably at least one of the two or more binding moieties is an antibody. The antibody can comprise a variable domain that binds to an extracellular part of EGFR and a variable domain that binds to an extracellular part of HER2. In another embodiment the antibody comprises a variable domain that binds to an extracellular part of EGFR and a variable domain that binds to an extracellular part of HER3.

The two or more binding moieties preferably comprise two or more antibodies, preferably multispecific antibodies that each comprise a variable domain that binds to an extracellular part of EGFR; and wherein a first of said antibodies comprises a variable domain that binds to an extracellular part of HER2 and a second of said antibodies comprises a variable domain that binds to an extracellular part of HER3. A preferred example of a composition comprising two or more multispecific antibodies is a composition comprising two or more bispecific antibodies. A non-limiting example of a composition comprising two bispecific antibodies as described herein is schematically depicted in FIG. 1. Two bispecific antibodies are depicted that each have two heavy chains (1) and two light chains (4). The two antibodies share a heavy chain with heavy chain variable region (5). They differ in the variable region of the other heavy chain. One antibody has heavy chain variable region (6). The other antibody has heavy chain variable region (7). All heavy chain variable regions can pair with the common light chain (4) to form functional binding domains. When produced by the same cell the heavy chains are directed to heterodimerize by the presence of a heterodimerization domain (2, 3). The heterodimerization domain has two parts, one part on one heavy chain and the compatible part on the other heavy chain. The heterodimerization domain is often in the IgG1 CH3 region. Heterodimerization can be directed by providing the appropriate part to selected heavy chains.

In the present invention selected formation of EGFR×HER2 and EGFR×HER3 bispecific antibodies may be directed by incorporating one part. (3) of the heterodimerization domain in the heavy chain the forms the EGFR variable domain and the compatible part (2) in the heavy chains that form the HER2 and the HER3 binding domains.

A heavy chain that has a heavy chain variable region that together with a light chain forms a variable domain that binds an antigen such as EGFR, HER2 or HER3 is herein also referred to as the EGFR heavy chain, or the HER2 heavy chain etc. In a preferred embodiment of the invention the CH3-regions of the heavy chains of a first and/or a second antibody are engineered to facilitate heterodimerization of a EGFR heavy chain with a HER2 heavy chain and a EGFR heavy chain with a HER3 heavy chain. In a preferred embodiment, the engineering to facilitate heterodimerization employs DEKK residue positions previously described in U.S. Pat. Nos. 9,248,182; 9,358,286; 9,248,182; and 9,758,805.

In some embodiments the binding of the antibodies of the composition to EGFR blocks the binding of EGF to EGFR and/or wherein the binding of antibodies of the composition to HER3 blocks the binding of neuregulin 1 (NRG) to HER3. In a preferred embodiment the binding of the antibodies of the composition to EGFR blocks the binding of EGF to EGFR and the binding of antibodies of the composition to HER3 blocks the binding of neuregulin 1 (NRG) to HER3

A variable domain that binds to an extracellular part of EGFR preferably comprises a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs.

A variable domain that binds to an extracellular part of HER2 preferably comprises a heavy chain variable region comprising a CDR1 sequence SYGMH, a CDR2 sequence VISYDGSNKYYADSVKG and a CDR3 sequence DYYRRTARAGFDY or a variant thereof comprising a substitution, deletion and/or insertion of 1, 2, or 3 amino acids in the CDRs.

A variable domain that binds to an extracellular part of HER3 preferably comprises a heavy chain variable region comprising a CDR1 sequence GYYMH, a CDR2 sequence WINPNSGGTNYAQKFQG and a CDR3 sequence DHGSRHFWSYWGFDY or a variant thereof comprising a substitution, deletion and/or insertion of 1, 2, or 3 amino acids in the CDRs.

In a preferred embodiment a composition comprises two bispecific antibodies wherein a first of said bispecific antibodies comprises a variable domain that binds to an extracellular part of EGFR comprising a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs. In a preferred embodiment a first and a second of said bispecific antibodies comprises a variable domain that binds to an extracellular part of EGFR comprising a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs.

In a preferred embodiment a first and a second of said bispecific antibodies comprises a variable domain that binds to an extracellular part of EGFR comprising a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs and wherein said first bispecific antibody further comprises a variable domain that binds to an extracellular part of HER2 which variable domain preferably comprises a heavy chain variable region comprising a CDR1 sequence SYGMH, a CDR2 sequence VISYDGSNKYYADSVKG and a CDR3 sequence DYYRRTARAGFDY or a variant thereof comprising a substitution, deletion and/or insertion of 1, 2, or 3 amino acids in the CDRs and wherein said second bispecific antibody further comprises a variable domain that binds to an extracellular part of HER3 which variable domain preferably comprises a heavy chain variable region comprising a CDR1 sequence GYYMH, a CDR2 sequence WINPNSGGTNYAQKFQG and a CDR3 sequence DHGSRHFWSYWGFDY or a variant thereof comprising a substitution, deletion and/or insertion of 1, 2, or 3 amino acids in the CDRs.

In a preferred embodiment a first and a second of said bispecific antibodies comprises a variable domain that binds to an extracellular part of EGFR comprising a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs and wherein said first bispecific antibody further comprises a variable domain that binds to an extracellular part of HER2 which variable domain preferably comprises a heavy chain variable region comprising a CDR1 sequence SYGMH, a CDR2 sequence VISYDGSNKYYADSVKG and a CDR3 sequence GDYGSYSSYAFDY or a variant thereof comprising a substitution, deletion and/or insertion of 1, 2, or 3 amino acids in the CDRs and wherein said second bispecific antibody further comprises a variable domain that binds to an extracellular part of HER3 which variable domain preferably comprises a heavy chain variable region comprising a CDR1 sequence GYYMH, a CDR2 sequence WINPNSGGTNYAQKFQG and a CDR3 sequence DHGSRHFWSYWGFDY or a variant thereof comprising a substitution, deletion and/or insertion of 1, 2, or 3 amino acids in the CDRs.

Conservative variations of 1, 2 or 3 amino acid residues from the recited CDR sequences are allowed while retaining the same kind of binding activity (in kind, not necessarily in amount). Hence, said heavy chain CDR 1, 2 and 3 sequences preferably contain sequences that deviate in no more than three, preferably no more than two, more preferably no more than one amino acid from the recited CDR sequences. In certain embodiments, the heavy chain CDR 1, 2 and 3 sequences are identical to the recited CDR sequences.

In some embodiments, the EGFR variable domain comprises a heavy chain variable region which comprises the HCDR1, HCDR2 and HCDR3 of an EGFR VH region set forth in FIG. 7 or FIG. 8. Preferably of MF3755 of FIG. 7 or FIG. 8.

In some embodiments, the EGFR variable domain comprises a heavy chain variable region which comprises an amino acid sequence at least 90%, preferably at least 95%, more preferably at least 97%, more preferably at least 98%, more preferably at least 99% identical or 100% identical to the amino acid sequence of an EGFR VH region set forth in FIG. 7 or FIG. 8. Preferably of MF3755 of FIG. 7 or FIG. 8.

For example, in some embodiments, the heavy chain variable region of the bispecific antibody that binds human EGFR can have 0-10, preferably 0-5 amino acid insertions, deletions, substitutions, additions in the sequence of the heavy chain variable region outside of the three CDR sequences, or a combination thereof. In some embodiments, the heavy chain variable region comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain embodiments, the EGFR variable domain comprises a heavy chain variable region comprising an amino acid sequence from an EGFR VH region selected from FIG. 7 or FIG. 8. Preferably of MF3755 of FIG. 7 or FIG. 8.

In some embodiments, the HER2 variable domain comprises a heavy chain variable region which comprises the HCDR1, HCDR2 and HCDR3 of a HER2 VH region set forth in FIG. 7 or FIG. 8, preferably of MF2032 of FIG. 7 or FIG. 8.

In some embodiments, the HER2 variable domain comprises a heavy chain variable region which comprises an amino acid sequence at least 90%, preferably at least 95%, more preferably at least 97%, more preferably at least 98%, more preferably at least 99% identical or 100% identical to the amino acid sequence of a HER2 VH region set forth in FIG. 7 or FIG. 8, preferably of MF2032 of FIG. 7 or FIG. 8.

For example, in some embodiments, the heavy chain variable region of the bispecific antibody that binds human HER2 can have 0-10, preferably 0-5 amino acid insertions, deletions, substitutions, additions in the sequence of the heavy chain variable region outside of the three CDR sequences, or a combination thereof. In some embodiments, the heavy chain variable region comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain embodiments, the HER2 variable domain comprises a heavy chain variable region comprising an amino acid sequence selected from MF1849 or of MF2032 of FIG. 7 or FIG. 8; preferably of MF2032 of FIG. 7 or FIG. 8.

In some embodiments, the HER3 variable domain comprises a heavy chain variable region which comprises the HCDR1, HCDR2 and HCDR3 of the VH region of MF3178 of FIG. 7 or FIG. 8.

In some embodiments, the HER3 variable domain comprises a heavy chain variable region which comprises an amino acid sequence at least 90%, preferably at least 95%, more preferably at least 97%, more preferably at least 98%, more preferably at least 99% identical or 100% identical to the amino acid sequence set forth in of MF3178 of FIG. 7 or FIG. 8.

For example, in some embodiments, the heavy chain variable region of the bispecific antibody that binds human HER3 can have 0-10, preferably 0-5 amino acid insertions, deletions, substitutions, additions in the sequence of the heavy chain variable region outside of the three CDR sequences, or a combination thereof. In some embodiments, the heavy chain variable region comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain embodiments, the HER3 variable domain comprises a heavy chain variable region comprising an amino acid sequence of MF3178 of FIG. 7 or FIG. 8.

In a preferred embodiment a first and a second of said bispecific antibodies comprises a variable domain that binds to an extracellular part of EGFR comprising a heavy chain variable region comprising an amino acid sequence from an EGFR VH region selected from FIG. 7 or FIG. 8 or a variant thereof, preferably of MF3755 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 which variable domain comprises a heavy chain variable region comprising an amino acid sequence selected from MF1849 or MF2032 of FIG. 7 or FIG. 8 or a variant thereof; preferably of MF2032 of FIG. 7 or FIG. 8 or a variant thereof wherein said variant comprises a substitution, deletion and/or insertion of 1, 2, or 3 amino acids that are not preferably not in the CDRs; and wherein said second bispecific antibody further comprises a variable domain that binds to an extracellular part of HER3 which variable domain comprises a heavy chain variable region comprising an amino acid sequence of MF3178 of FIG. 7 or FIG. 8 or a variant thereof wherein said variant comprises a substitution, deletion and/or insertion of 1, 2, or 3 amino acids that are not preferably not in the CDRs.

In a preferred embodiment a first and a second of said bispecific antibodies comprises a variable domain that binds to an extracellular part of EGFR comprising a heavy chain variable region comprising an amino acid sequence of MF3755 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 which variable domain comprises a heavy chain variable region comprising an amino acid sequence of MF2032 of FIG. 7 or FIG. 8 or a variant thereof comprising a substitution, deletion and/or insertion of 1, 2, or 3 amino acids that are not preferably not in the CDRs; and wherein said second bispecific antibody further comprises a variable domain that binds to an extracellular part of HER3 which variable domain comprises a heavy chain variable region comprising an amino acid sequence of MF31.78 of FIG. 7 or FIG. 8 or a variant thereof wherein said variant comprises a substitution, deletion and/or insertion of 1, 2, or 3 amino acids that are not preferably not in the CDRs.

In one embodiment a first and a second of said bispecific antibodies comprises a variable domain that binds to an extracellular part of EGFR comprising a heavy chain variable region comprising an amino acid sequence of MF4280 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 comprising a heavy chain variable region comprising an amino acid sequence of MF1849 of FIG. 7 or FIG. 8 or a variant thereof comprising a substitution, deletion and/or insertion of 1, 2, or 3 amino acids that are not preferably not in the CDRs; and wherein said second bispecific antibody further comprises a variable domain that binds to an extracellular part of HER3 comprising a heavy chain variable region comprising an amino acid sequence of MF3178 of FIG. 7 or FIG. 8 or a variant thereof comprising a substitution, deletion and/or insertion of 1, 2, or 3 amino acids that are not preferably not in the CDRs.

In one embodiment a first and a second of said bispecific antibodies comprises a variable domain that binds to an extracellular part of EGFR comprising a heavy chain variable region comprising an amino acid sequence of MF4280 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 comprising a heavy chain variable region comprising an amino acid sequence of MF2032 of FIG. 7 or FIG. 8 or a variant thereof comprising a substitution, deletion and/or insertion of 1, 2, or 3 amino acids that are not preferably not in the CDRs; and wherein said second bispecific antibody further comprises a variable domain that binds to an extracellular part of HER3 comprising a heavy chain variable region comprising an amino acid sequence of MF3178 of FIG. 7 or FIG. 8 or a variant thereof comprising a substitution, deletion and/or insertion of 1, 2, or 3 amino acids that are not preferably not in the CDRs.

In one embodiment a first and a second of said bispecific antibodies comprises a variable domain that binds to an extracellular part of EGFR comprising a heavy chain variable region comprising an amino acid sequence of MF4003 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 comprising a heavy chain variable region comprising an amino acid sequence of MF1849 of FIG. 7 or FIG. 8 or a variant thereof comprising a substitution, deletion and/or insertion of 1, 2, or 3 amino acids that are not preferably not in the CDRs; and wherein said second bispecific antibody further comprises a variable domain that binds to an extracellular part of HER3 comprising a heavy chain variable region comprising an amino acid sequence of MF3178 of FIG. 7 or FIG. 8 or a variant thereof comprising a substitution, deletion and/or insertion of 1, 2, or 3 amino acids that are not preferably not in the CDRs.

In one embodiment a first and a second of said bispecific antibodies comprises a variable domain that binds to an extracellular part of EGFR comprising a heavy chain variable region comprising an amino acid sequence of MF4003 of FIG. 7 or FIG. 8 or a variant thereof comprising a substitution, deletion and/or insertion of 1, 2, or 3 amino acids that are not preferably not in the CDRs; and wherein said first bispecific antibody further comprises a variable domain that binds to an extracellular part of HER2 comprising a heavy chain variable region comprising an amino acid sequence of MF2032 of FIG. 7 or FIG. 8 or a variant thereof comprising a substitution, deletion and/or insertion of 1, 2, or 3 amino acids that are not preferably not in the CDRs; and wherein said second bispecific antibody further comprises a variable domain that binds to an extracellular part of HER3 comprising a heavy chain variable region comprising an amino acid sequence of MF3178 of FIG. 7 or FIG. 8 or a variant thereof comprising a substitution, deletion and/or insertion of 1, 2, or 3 amino acids that are not preferably not in the CDRs.

Exemplary EGFR heavy chain variable regions are described in WO2015/130172 and PCT/NL2018/050537 which are incorporated by reference herein. Exemplary HER2 heavy chain variable regions are described in WO2015/130173 which is incorporated by reference herein. Exemplary HER3 heavy chain variable regions are described in WO2015/130172; and WO2015/130173 which are incorporated by reference herein.

Additional variants of the disclosed amino acid sequences which retain EGFR, HER2 or HER3 binding can be obtained, for example, from phage display libraries which contain the rearranged human IGKV1-39/IGKJ1 VL region (De Kruif et al. Biotechnol Bioeng. 2010 (106741-50), and a collection of VH regions incorporating amino acid substitutions into the amino acid sequence of an EGFR. HER2 or HER3 VH region disclosed herein, as previously described. Phages encoding Fab regions which bind EGFR, HER2 or HER3 may be selected and analyzed by flow cytometry, and sequenced to identify variants with amino acid substitutions, insertions, deletions or additions which retain antigen binding.

The invention further provides a binding moiety that specifically binds an extracellular part of EGFR and an extracellular part of HER2. The binding moiety preferably comprises a variable domain that binds EGFR and a variable domain that binds HER2. The variable domain that binds EGFR is preferably an EGFR variable domain as described herein. The variable domain that binds HER2 is preferably a HER2 variable domain as described herein. Preferably both of the EGFR and the HER2 variable domains are variable domains as described herein.

The invention further provides a binding moiety that specifically binds an extracellular part of EGFR and an extracellular part of HER3. The binding moiety preferably comprises a variable domain that binds EGFR and a variable domain that binds HER3. The variable domain that binds EGFR is preferably an EGFR variable domain as described herein. The variable domain that binds HER3 is preferably a HER3 variable domain as described herein. Preferably both of the EGFR and the HER3 variable domains are variable domains as described herein.

The invention further provides a composition comprising a binding moiety that specifically binds an extracellular part of EGFR and an extracellular part of HER2 and a binding moiety that specifically binds an extracellular part of EGFR and an extracellular part of HER3.

A binding moiety as described herein is preferably an antibody, preferably a multispecific antibody, preferably a bispecific antibody.

The light chain variable regions (VIA) of the EGFR variable domain, the HER2 variable domain and the HER3 variable domain of the binding moieties such as the bispecific antibodies may be the same as the VL region of parental EGFR monospecific antibody; the VL region of parental HER2 monospecific antibody, and/or the same as the parental HER3 monospecific antibody. Alternative VL regions may be used for one or more of the VH/VL region combinations as long as the variable domains retain binding to respectively EGFR, HER2 or HER3.

In some embodiments, the VL region of the EGFR variable domain, the HER2 variable domain and the HER3 variable domain are similar. In certain embodiments, VL regions in all of the variable domains of the binding moieties are identical.

In certain embodiments, the light chain variable region of one, two, three or more variable domains of the binding moieties of the invention comprise a common light chain variable region. In some embodiments, the common light chain variable region of one, two, three or more variable domains comprise a germline variable region V-segment. In certain embodiment, the light chain variable region of one, two, three or more variable domains comprise the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740: Entrez Gene: 28930; Ensembl: ENSG00000242371. The amino acid sequence for the V-region is provided in sequence 10 of FIG. 7. The V-region can be combined with one of five J-regions. Preferred J-regions are jk1, and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain embodiments, the light chain variable region of one or both VH/VL binding regions comprises the kappa light chain IgVκ1-39*01/IGJκ1*01 or IgVκ1-39*01/IGJκ1*05 (Sequence 11 or sequence 12 of FIG. 7, respectively).

In some embodiments, the light chain variable region of one, two, three or more variable domains of the binding moieties of the invention comprise an LCDR1 comprising the amino acid sequence QSISSY (Sequence 7 of FIG. 7), an LCDR2 comprising the amino acid sequence AAS, and an LCDR3 comprising the amino acid sequence QQSYSTP (Sequence 9 of FIG. 7) (i.e., the CDRs of IGKV1-39 according to IMGT). In some embodiments, the light chain variable region of one, two, three or more variable domains of binding moieties of the invention comprise an LCDR1 comprising the amino acid sequence QSISSY (Sequence 7 of FIG. 7), an LCDR2 comprising the amino acid sequence AASLQS (Sequence 8 of FIG. 7), and an LCDR3 comprising the amino acid sequence QQSYSTP (Sequence 9 of FIG. 7).

In some embodiments one, two, three or more variable domains of binding moieties of the invention comprise a light chain variable region comprising an amino acid sequence that is at least 90%, preferably at least 95%, more preferably at least 97%, more preferably at least 98%, more preferably at least 99% identical or 100% identical to the amino acid sequence of set forth in Sequence 11 of FIG. 7. In some embodiments, one, two, three or more variable domains of binding moieties of the invention comprise a light chain variable region comprising an amino acid sequence that is at least 90%, preferably at least 95%, more preferably at least 97%, more preferably at least 98%, more preferably at least 99% identical or 100% identical to the amino acid sequence of set forth in Sequence 11 of FIG. 7.

For example, in some embodiments, the variable light chain of one, two, three or more variable domains of the binding moieties of the invention can have from 0 to 10, preferably from 0 to 5 amino acid insertions, deletions, substitutions, additions or a combination thereof with respect to Sequence 11 of FIG. 7 or Sequence 12 of FIG. 7. In some embodiments, the light chain variable region of one, two, three or more variable domains of the binding moieties of the invention comprise from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other embodiments, the light chain variable region of one, two, three or more variable domains of the binding moieties of the invention, comprises the amino acid sequence of Sequence 11 of FIG. 7 or Sequence 12 of FIG. 7. In certain embodiments, all variable domains of the binding moieties of the invention comprise identical VL regions. In one embodiment, the VL of all variable domains of the binding moieties of the invention comprises the amino acid sequence set forth in Sequence 11 of FIG. 7. In one embodiment, the VL of all variable domains of the binding moieties of the invention comprises the amino acid sequence set forth in Sequence 12 of FIG. 7 or Sequence 12 of FIG. 7.

Multispecific antibodies such as bispecific antibodies disclosed herein can be provided in a number of formats. Many different formats of multispecific antibodies are known in the art, and have been reviewed by Kontermann (Drug Discov Today, 2015 July; 20(7):838-47; MAbs, 2012 March-April; 4(2):182-97) and in Spiess et al., (Alternative molecular formats and therapeutic applications for bispecific antibodies. Mol. Immunol. (2015) http://dx.doi.org/10.1016/j.molimm.2015.01.003), which are each incorporated herein by reference. For example, multispecific antibody formats such as bispecific antibody formats that are not classical antibodies with two variable domains, have at least a variable domain comprising a heavy chain variable region and a light chain variable region. This variable domain may be linked to a single chain Fv-fragment, monobody, a VHH and a Fab-fragment that provides a second binding activity.

In some embodiments, the multispecific antibodies used in the methods provided herein are generally of the human IgG subclass (e.g., for instance IgG1, IgG2, IgG3, IgG4). In certain embodiments, the antibodies are of the human IgG1 subclass. Full length IgG antibodies are preferred because of their favorable half-life and for reasons of low immunogenicity. Such multispecific antibodies may have two different heavy chains comprising a heterodimerization domain. Accordingly, in certain embodiments, the EGFR/HER2 and EGFR/HER3 bispecific antibodies are full length IgG molecules. In an embodiment, the EGFR/HER2 and EGFR/HER3 bispecific antibodies are full length IgG1 molecules.

Accordingly, in certain embodiments, the multispecific EGFR/HER2 and EGFR/HER3 antibodies comprise a fragment crystallizable (Fe). The Fe regions of the multispecific antibodies are preferably comprised of a human constant region. A constant region or Fc of the multispecific antibodies may contain one or more, preferably not more than 10, preferably not more than 5 amino-acid differences with a constant region of a naturally occurring human antibody. For example, in certain embodiments, each Fab-arm of the bispecific antibodies may further include an Fe-region comprising modifications promoting the formation of the bispecific antibody, modifications affecting Fe-mediated effector functions, and/or other features described herein.

In preferred embodiments, the multispecific, preferably bispecific full length IgG antibody have a lower hinge and/or CH2 domains such that interaction of said bispecific IgG antibody with Fc gamma (Fcγ) receptors is enhanced. Antibody-dependent cellular cytotoxicity also referred to as ADCC activity of an antibody can often be improved when the antibody itself has a low ADCC activity. This is for instance achieved by removing fucose residues from glycosylated part of the antibody. One technique for enhancing ADCC by afucosylation is described in for instance Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). A multispecific antibody as described herein is preferably afucosylated. Preferably both multispecific antibodies are afucosylated. Other strategies have been reported to achieve ADCC enhancement, for instance including glycoengineering (Kyowa Hakko/Biowa, GlycArt (Roche) and Eureka Therapeutics) and mutagenesis (Xencor and Macrogenics), all of which seek to improve Fc binding to low-affinity activating FcγRIIIa, and/or to reduce binding to the low affinity inhibitory FcγRIIb.

Bispecific antibodies are typically produced by cells that express nucleic acid(s) encoding the antibody. Accordingly, in some embodiments, a method for producing a composition comprising a multispecific antibody that binds EGFR and HER2 and a multispecific antibody that binds EGFR and HER3 is provided comprising providing a cell with
a nucleic acid that encodes a polypeptide comprising a heavy chain that together with a common light chain forms a variable domain that binds to an extracellular part of EGFR;
a nucleic acid that encodes a polypeptide comprising a heavy chain that together with said common light chain forms a variable domain that binds to an extracellular part of HER2;
a nucleic acid that encodes a polypeptide comprising a heavy chain that together with said common light chain forms a variable domain that binds to an extracellular part of HER3; and
a nucleic acid that encodes a polypeptide comprising said common light chain;
   wherein two or more of said nucleic acids may be physically linked or not and wherein each of said nucleic acids further comprises an expression control sequence to allow expression of the encoded heavy and light chains in said cell and wherein the method further comprises culturing said cell to allow expression of said heavy and light chains and, optionally, collecting said two or more antibodies. The two or more said antibodies may be collected from the cells and/or the supernatant.

The level at which the respective chains are produced in a cell can be tailored, for instance by selecting appropriate expression control sequences or by selecting the number of introduced copies of nucleic acid or both. In a preferred embodiment a collection of cells is provided with said nucleic acid and a clone is selected that expresses the appropriate levels of the respective chains. The clone is typically also selected on the basis of the amount of antibodies produced. In one embodiment said method comprises providing a collection of cells with said nucleic acid and selecting from said collection a cell with a desired ratio of expression of the respective heavy and light chains. In some embodiments said two or more binding moieties are antibodies, preferably bispecific antibodies. In some embodiments the cells preferably produce essentially equimolar amounts of the two or more binding moieties. In other embodiments the cells produce more of one binding moiety than of another of said two or more binding moieties.

The invention also provides a cell comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with said common light chain forms a variable domain that binds to an extracellular part of HER2;
a nucleic acid that encodes a polypeptide comprising a heavy chain that together with said common light chain forms a variable domain that binds to an extracellular part of HER3; and
a nucleic acid that encodes a polypeptide comprising said common light chain;
   wherein two or more of said nucleic acids may be physically linked or not and wherein each of said nucleic acids further comprises an expression control sequence to allow expression of the encoded heavy and light chains in said cell.

The invention further provides a container comprising nucleic acid comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with a common light chain forms a variable domain that binds to an extracellular part of HER2;
a nucleic acid that encodes a polypeptide comprising a heavy chain that together with a common light chain forms a variable domain that binds to an extracellular part of HER3; and
a nucleic acid that encodes a polypeptide comprising said common light chain;
   wherein two or more of said nucleic acids may be physically linked or not and wherein each of said nucleic further comprises an expression control sequence to allow expression of the encoded heavy and light chains in a cell.

The cell that produces the binding moieties is preferably an animal cell more preferably a mammalian cell, more preferably a primate cell, most preferably a human cell. A suitable cell is any cell capable of comprising and preferably of producing the binding moieties, preferably the multispecific antibodies and preferably the bispecific antibodies as described herein.

Suitable cells for antibody production are known in the art and include a hybridoma cell, a Chinese hamster ovary (CHO) cell, an NS0 cell, an HER293 cell, a 293-F cell or a PER-C6 cell. Various institutions and companies have developed cell lines for the large scale production of antibodies, for instance for clinical use. Non-limiting examples of such cell lines are CHO cells, NS0 cells or PER.C6 cells. In a particularly preferred embodiment said cell is a human cell. Preferably a cell that is transformed by an adenovirus E1 region or a functional equivalent thereof. in a particularly preferred embodiment said cell is a CHO cell or a variant thereof. Preferably a variant that makes use of a Glutamine synthetase (GS) vector system for expression of an antibody. In one preferred embodiment, the cell is a CHO cell.

In some embodiments, the cell expresses three different heavy chains and at least one light chain. In one preferred embodiment, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1 39/IGKJ1 (huVκ1 39) light chain. The huVκ1 39 was previously shown to be able to pair with more than one heavy chain thereby giving rise to antibodies with diverse specificities, which facilitates the generation of bispecific molecules (WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred embodiment the bispecific antibody of the invention comprises two different immunoglobulin heavy chains with compatible heterodimerization domains. Various compatible heterodimerization domains have been described in the art. The compatible heterodimerization domains are preferably compatible immunoglobulin heavy chain CH3 heterodimerization domains. The art describes various ways in which such hetero-dimerization of heavy chains can be achieved.

One preferred method for producing the multispecific antibodies as described herein is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains. Introducing a further heavy chain that has either the DE- or the KK-variant heavy chain, allows the production of a further DEKK bispecific molecule. A newly introduced DE-heavy chain (DE²) can associate with the existing KK heavy chain. The cell thus produces two bispecific antibodies a DE¹KK and a DE²KK bispecific antibody. If a new KR heavy chain (KK²) is introduced instead of the new DE heavy chain, the bispecific antibodies with the combinations DEKK¹ and DEKK² are produced. The levels at which the different antibodies can be produced by the cell may be adjusted by adjusting the relative expression of the HER2 and HER3 chains with respect to each other. The light chain is typically produced sufficiently to reduce the level of single heavy chains and the level at which the EGFR chains is produced is typically sufficient to allow efficient pairing with the HER2, HER3 chains.

Accordingly, in one embodiment the heavy chain/light chain combination that comprises the variable domain that binds EGFR, comprises a DE variant of the heavy chain. In this embodiment the heavy chain/light chain combination that comprises the variable domain that binds HER2 and the heavy chain/light chain combination that comprises the variable domain that binds HER3 comprises a KK variant of the heavy chain.

A candidate EGFR/HER2 or EGFR/HER3 IgG bispecific antibody can be tested for binding using any suitable assay. For example, binding to membrane-expressed EGFR, HER2 or HER3. This is typically done on a cell that normally does not express the EGFR, HER2 or HER3 and that is transformed to express one of EGFR, HER2 or HER3. Binding of the antibody to the transformed cell and not to the untransformed cell is indicative for the specific binding of the antibody. Binding can be asses by, for instance, flow cytometry (according to the FACS procedure as previously described in WO2015/130172. PCT/NL2018/050537; and WO2015/130173. The respective monospecific antibodies can be taken along as controls as well as an irrelevant IgG1 isotype control mAb.

Binding moieties such as antibodies can be collected from the cells and/or the supernatant of a cell culture. Typically they are collected from the supernatant of the producing cells. Binding moieties such as antibodies can be purified from the supernatant. Many purification methods are known in the art. Some more common methods employ affinity purification.

Antibodies produced by a cell can be purified by affinity purification. This is advantageously done by means of protein A extraction. Eluted antibodies can be tested by ELISA for the presence of the specific binding properties, (i.e. binding to EGFR, to HER2 and HER3). The antibody preparation can further be analysed by ion-exchange column chromatography. The individual bispecific antibodies can be purified from each other by routine techniques, for example using ion exchange chromatography. The presence of the respective bispecific antibodies can also be analysed by ELISA. Binding of the preparation to HER2 and washing should remove all EGFR/HER3 antibodies. Staining with labelled soluble HER3 does not give a signal whereas staining with labelled soluble EGFR does. Binding of the preparation to HER3 and washing should remove all EGFR/HER2 antibodies. Staining with labelled soluble HER2 does not give a signal whereas staining with labelled soluble EGFR does. Binding of the preparation to EGFR and washing should not remove EGFR/HER2 and EGFR/HER3 antibodies. Staining with labelled soluble HER2 as well as staining with labelled soluble HER3 should give a signal. The levels of the respective antibodies in a preparation can, with the appropriate controls with known levels of the single bispecific antibody, also be estimated using such an ELISA.

A method for producing a composition comprising two or more bispecific antibodies comprises
providing cells with nucleic acid that encodes the bispecific antibodies;
culturing said cells;
harvest clarification;
collecting the bispecific antibodies from the culture; and
separating produced bispecific antibodies from half antibodies by ion exchange chromatography (IEX);
   the method characterized in that the bispecific antibodies exhibit similar IEX retention times, preferably that that deviate by 10% or less from the average of the retention times of the individual antibodies under the IEX conditions used. In one embodiment the antibodies are selected to have IEX retention times that that deviate by 10% or less from the average of the retention times of the individual antibodies under the IEX conditions used. The antibodies may be first purified from other proteins in the culture. This is typically done by means of affinity purification, preferably by protein A extraction. The bispecific antibodies are preferably selected to have half antibodies with retention times that are outside the range spanned by the retention times of the antibodies. Where combinations of bispecific antibodies are produced and monospecific antibodies are not desired, the bispecific antibodies are preferably selected to have retention times that are different from the retention times of the monospecific antibodies. The retention times of the monospecific antibodies in this embodiment are preferably outside the range spanned by the retention times of the respective bispecific, antibodies. Cells in the culture preferably express the three heavy chains simultaneously wherein the heavy chains comprises CH3 heterodimerization domains that facilitate the formation of EGFR/HER2 and EGFR/HER3 heavy chain heterodimerization. The cells preferably express a common light chain of FIG. 7. The bispecific antibodies in one embodiment have isoelectric points (PI) that are similar, and preferably do not differ by more than 0.5 units from the average PI of said at least two bispecific antibodies.

The affinities of the EGFR, HER2 and HER3 FABs of a candidate EGFR/HER2 or EGFR/HER bispecific antibody for their targets can be measured by surface plasmon resonance (SPR) technology using a BIAcore T100. An anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human EGFR-Fc, HER2-Fc and HER3-Fc protein are run over the sensor surface in a concentration range to measure on- and off-rates. After each cycle, the sensor surface is regenerated by a pulse of HCl and the bsAb is captured again. From the obtained sensorgrams, on- and off-rates and affinity values for binding to human EGFR, HER2 and HER3 are determined using the BIAevaluation software.

The invention also provides a composition as described herein for use in the treatment of cancer. In embodiments the cancer is a solid epithelial cancer. Preferably the composition is used for a cancer that expresses EGFR, HER2 and/or HER3. The composition is used for preferably for pancreatic cancer, colorectal cancer, head & neck cancer, epithelial ovarian cancer, epithelial fallopian tube cancer, epithelial peritoneal cancer, bladder cancer, or prostate cancer. In embodiments the cancer treated by use of the composition is advanced cancer. The composition is used for preferably for metastatic cancer. The composition is used for preferably for metastatic pancreatic cancer, metastatic colorectal cancer, metastatic head & neck cancer, metastatic epithelial ovarian cancer, metastatic epithelial fallopian tube cancer, metastatic epithelial peritoneal cancer, metastatic bladder cancer, or metastatic prostate cancer. In embodiments, the composition is used for preferably for the cancer that is gastric cancer, lung cancer, breast cancer or esophagus cancer. Preferably, the composition is used for metastatic gastric cancer, metastatic lung cancer, metastatic breast cancer or metastatic esophagus cancer.

The invention further provides two or more binding moieties that each comprise a variable domain that binds to an extracellular part of EGFR; wherein a first of said binding moieties comprises a variable domain that binds to an extracellular part of HER2 and a second of said binding moieties comprises a variable domain that binds to an extracellular part of HER3 for use in the treatment of cancer. Also provided is a product containing two or more binding moieties that each comprise a variable domain that binds to an extracellular part of EGFR; wherein a first of said binding moieties comprises a variable domain that binds to an extracellular part of HER2 and a second of said binding moieties comprises a variable domain that binds to an extracellular part of HER3 as a combined preparation for simultaneous, separate or sequential use in treating cancer.

The cancer treated by embodiments of the invention is preferably a cancer as indicated elsewhere herein. The cancer preferably comprises cells with an EGFR-mutation that renders the cell resistant to treatment with a tyrosine kinase inhibitor (TKI). In some embodiments the cancer comprises cells with an EGFR R521K polymorphism. The cancer treated and method of treatment of an invention described herein is preferably gastric cancer, lung cancer or oesophagus cancer. In a further embodiment the invention provides a method for the treatment of a subject that has cancer or is at risk of recurrence, relapse of cancer, the method comprising administering the subject in need thereof two or more binding moieties that each comprise a variable domain that binds to an extracellular part of EGFR; wherein a first of said binding moieties comprises a variable domain that binds to an extracellular part of HER2 and a second of said binding moieties comprises a variable domain that binds to an extracellular part of HER3.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having a cancer).

The terms “treat.” “treating.” and “treatment,” as used herein, refer to any type of intervention or process performed on, or administering an active agent or combination of active agents to the subject with the objective of reversing. alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of caner. Effective treatment may, for example, decrease in tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of a composition of the invention, to effect a decrease in a caner (for example a decrease in the number of cancer cells) or slowing of progression of a cancer. An effective amount of the combination therapy is administered according to the methods described herein in an “effective regimen” which refers to a combination of the binding moieties as indicated herein, wherein the order of administration and dosage frequency is adequate to effect treatment.

As used herein, the terms “synergy”. “therapeutic synergy”, and “synergistic effect” refer to a phenomenon where treatment of patients with a combination of binding moieties as indicated herein (e.g., a composition comprising a binding moiety that binds EGFR and HER2 and a binding moiety that binds EGFR and HER3) manifests a therapeutically superior outcome to the outcome achieved by each individual constituent of the combination when used alone (see, e.g., T. H. Corbett et al., 1982, Cancer Treatment Reports, 66, 1187). In this context a therapeutically superior outcome includes one or more of the following (a) an increase in therapeutic response that is greater than the sum of the separate effects of each binding moiety alone at the same dose as in the combination; (b) a decrease in the dose of one or more agents in the combination without a decrease in therapeutic efficacy; (c) a decrease in the incidence of adverse events while receiving a therapeutic benefit that is equal to or greater than the monotherapy of each agent at the same dose as in the combination, (d) a reduction in dose-limiting toxicities while receiving a therapeutic benefit that is greater than the monotherapy of each agent; (e) a delay or minimization of the induction of drug resistance.

In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone. Synergism of a drug combination may be determined, for example, according to the combination index (CI) theorem of Chou-Talalay (Chou et al., Adv. Enzyme Regul. 1984; 22:27-55; Chou, Cancer Res. 2010; 70(2):440-446).

The invention further provides a composition of the invention for use in the treatment of cancer. The embodiment used preferably treats a gastric cancer, colorectal cancer, colon cancer, gastro-esophageal cancer, esophageal caner, endometrial cancer, ovarian cancer, liver cancer, lung cancer including non-small cell lung cancer, clear cell sarcoma, salivary gland cancer, head and neck cancer, brain cancer, bladder cancer, pancreatic cancer, prostate cancer, kidney cancer, skin cancer, melanoma, and the like. In one embodiment the embodiment treats a cancer that is gastric cancer, lung cancer or esophagus cancer. The use preferably treats a cancer that is gastric cancer.

An invention described herein applies to the treatment of a cancer that is preferably a cancer that is tested for the presence of EGFR, HER2 and/or HER3 on the cell membrane. This can be done by routine methods and is typically analysed by immunohistochemistry.

The cancer preferably expresses HER2. The cancer preferably also expresses EGFR or HER3. The cancer preferably expresses EGFR. The cancer preferably also expresses HER2 or HER3. The cancer preferably expresses HER3. The cancer preferably also expresses EGFR or HER2. In some embodiments cells of the cancer and/or stromal cells in the cancer treated by the invention disclosed herein express an EGFR ligand, a HER3 ligand or both. Expression of the ligand and the receptor therefore may provide a growth stimulus to cells of the cancer. A combination of the invention is particularly suited to the treat cancers comprising such cells.

Expression of one of EGER, HER2 and HER3 in a treatment of the invention can at least delay escape of some of the tumors. Tumors that are targeted with a monospecific therapy can escape treatment by starting to express another of EGFR, HER2 or HER3 or by expressing a ligand for a receptor.

Such cells, if they occur, are also attacked by the binding moieties of the invention and can therefore be removed before they grow out and diversify themselves. In one embodiment the cancer is tested for the presence of a mutated EGFR. Many EGFR-positive tumors have a mutation in the gene that renders the cells resistant to treatment with a tyrosine kinase inhibitor.

A composition of the invention is suited to treat a cancer with an EGFR-mutation that renders the cancer cells resistant to treatment with a tyrosine kinase inhibitor (TKI). In one embodiment the cancer comprises cells with an EGFR R521K polymorphism. In some embodiments the caner is known to be resistant to first generation TKI inhibitors such as gefitinib and erlotinib.

A treatment of cancer as indicated herein can be combined with a further treatment of the cancer. Such a treatment may comprise a further binding moiety such as an antibody and/or a cytostatic drug, or protein kinase inhibitor. The protein kinase inhibitor is preferably an inhibitor other than an EGFR or HER3 tyrosine kinase inhibitor. Non-limiting examples of further treatments comprise radiotherapy, chemotherapy, surgery, vascular growth inhibition therapy and heat therapy.

A composition of the invention may be suited for use in the treatment of a cancer which is resistant to EGFR inhibition, wherein EGFR resistance is a result of over-expression of HER2 and/or HER3.

A composition of the invention may be suited for use in the treatment of a cancer which is resistant to HER2 inhibition wherein HER2 resistance is a result of over-expression of EGFR and/or HER3.

A composition of the invention may be suited for use in the treatment of a cancer which is resistant to HER3 inhibition wherein HER3 resistance is a result of over-expression of EGFR and/or HER2.

The term “Oligoclonics®” in the context of antibodies, binding moieties, compositions or products as described herein refers to the presence of more than one and typically 10 or less different antibodies or binding moieties in one preparation, including the presence of a bispecific. An example of Oligoclonics® includes a combination of two bispecific antibodies.

The invention further provides a binding moiety or bispecific antibody comprising a variable domain that binds to an extracellular part of EGFR and a variable domain that binds an extracellular part of HER2; wherein the EFGR variable domain comprises a heavy chain variable region comprising the CDRs of the heavy chain variable region MF3755, of MF4280, of MF4003 or of MF4016 of FIG. 8 or a variant thereof comprising a substitution, deletion and/or insertion of 1, 2, or 3 amino acids in the CDRs and wherein the HER2 variable domain comprises a heavy chain variable region comprising the CDRs of the heavy chain variable region MF2032 or MF1849 or a variant thereof comprising a substitution, deletion and/or insertion of 1, 2, or 3 amino acids in the CDRs.

Also provided is a binding moiety or bispecific antibody comprising a variable domain that binds to an extracellular part of EGFR and a variable domain that binds an extracellular part of HER2; wherein the EFGR variable domain comprises a heavy chain variable region comprising the CDRs of the heavy chain variable region MF3755 of FIG. 8 or a variant thereof comprising a substitution, deletion and/or insertion of 1, 2, or 3 amino acids in the CDRs and wherein the HER2 variable domain comprises a heavy chain variable region comprising the CDRs of the heavy chain variable region MF2032 or a variant thereof comprising a substitution, deletion and/or insertion of 1, 2, or 3 amino acids in the CDRs.

The invention further provides a binding moiety or bispecific antibody comprising a variable domain that binds to an extracellular part of EGFR and a variable domain that binds an extracellular part of HER2; wherein the EFGR variable domain comprises a heavy chain variable region comprising the amino acid sequence of the heavy chain variable region MF3755, of MF4280, of MF4003 or of MF4016 of FIG. 8 or a variant thereof comprising a substitution, deletion and/or insertion of 1, 2, or 3 amino acids that are not preferably not in the CDRs and wherein the HER2 variable domain comprises a heavy chain variable region comprising the amino acid sequence of the heavy chain variable region MF2032 or MF1849 or a variant thereof comprising a substitution, deletion and/or insertion of 1, 2, or 3 amino acids that are not preferably not in the CDRs.

The invention further provides a binding moiety or bispecific antibody comprising a variable domain that binds to an extracellular part of EGFR and a variable domain that binds an extracellular part of HER2; wherein the EFGR variable domain comprises a heavy chain variable region comprising the amino acid sequence of the heavy chain variable region MF3755 of FIG. 8 or a variant thereof comprising a substitution, deletion and/or insertion of 1, 2, or 3 amino acids that are not preferably not in the CDRs and wherein the HER2 variable domain comprises a heavy chain variable region comprising the amino acid sequence of the heavy chain variable region MF2032 or a variant thereof comprising a substitution, deletion and/or insertion of 1, 2, or 3 amino acids that are not preferably not in the CDRs.

Also provided is a binding moiety or bispecific antibody comprising a variable domain that binds to an extracellular part of EGFR and a variable domain that binds an extracellular part of HER2; wherein the EFGR variable domain comprises a heavy chain variable region comprising the CDRs of the heavy chain variable region MF3755 of FIG. 8 or a variant thereof comprising a substitution, deletion and/or insertion of 1, 2, or 3 amino acids in the CURS and wherein the HER2 variable domain comprises a heavy chain variable region comprising the CDRs of the heavy chain variable region MF1849 or a variant thereof comprising a substitution, deletion and/or insertion of 1, 2, or 3 amino acids in the CDRs.

The invention further provides a binding moiety or bispecific antibody comprising a variable domain that binds to an extracellular part of EGFR and a variable domain that binds an extracellular part of HER2; wherein the EFGR variable domain comprises a heavy chain variable region comprising the amino acid sequence of the heavy chain variable region MF3755 of FIG. 8 or a variant thereof comprising a substitution, deletion and/or insertion of 1, 2, or 3 amino acids that are not preferably not in the CDRs and wherein the HER2 variable domain comprises a heavy chain variable region comprising the amino acid sequence of the heavy chain variable region MF1849 or a variant thereof comprising a substitution, deletion and/or insertion of 1, 2, or 3 amino acids that are not preferably not in the CDRs.

For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1

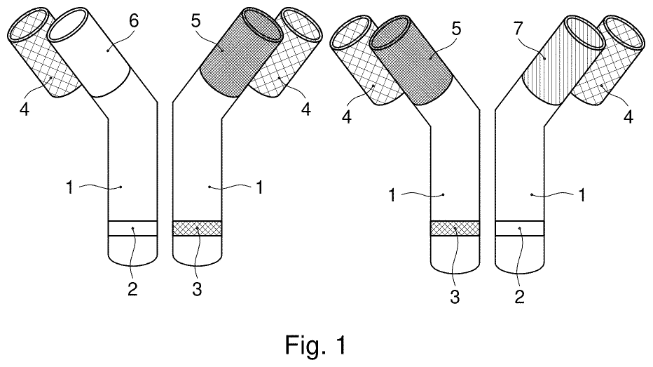

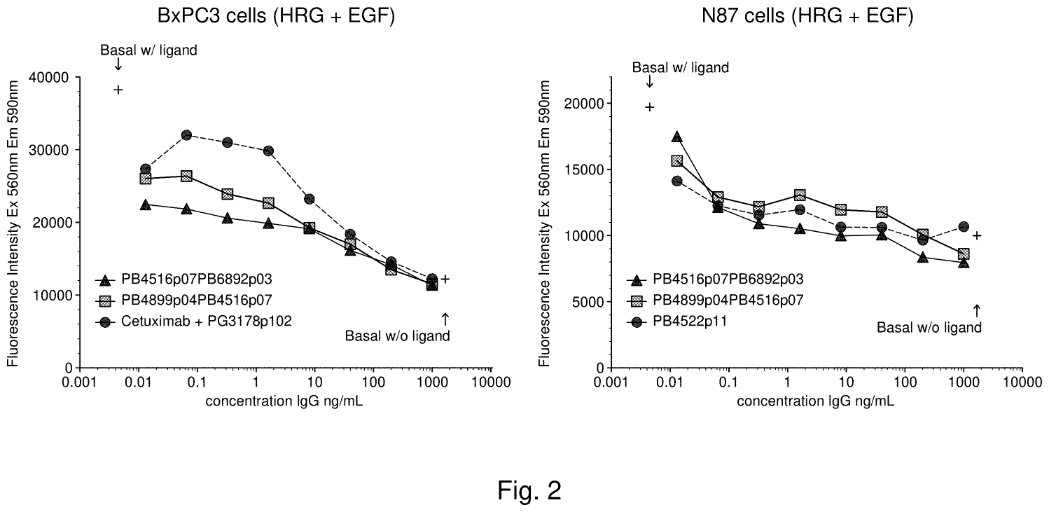

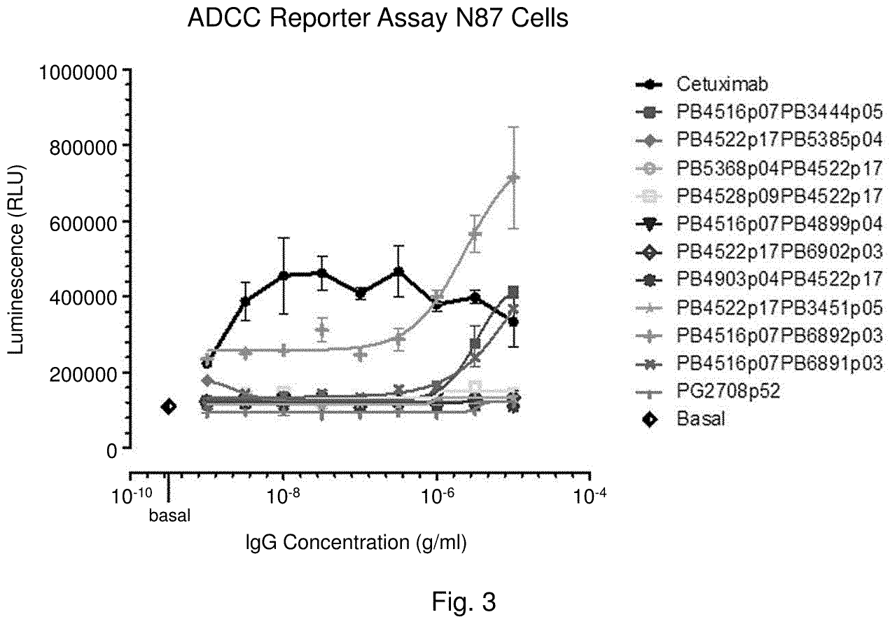

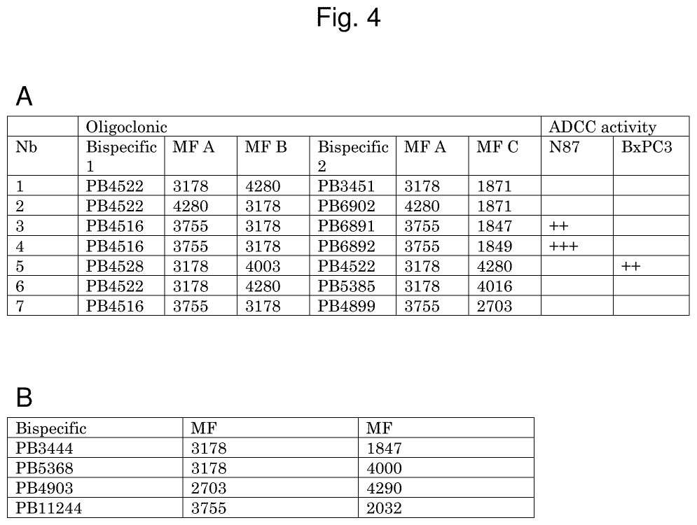

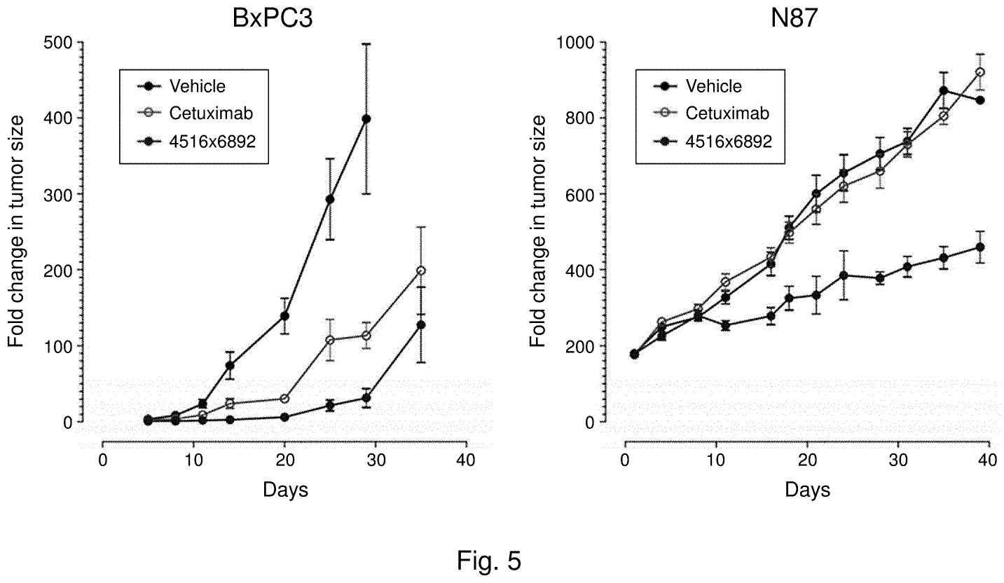

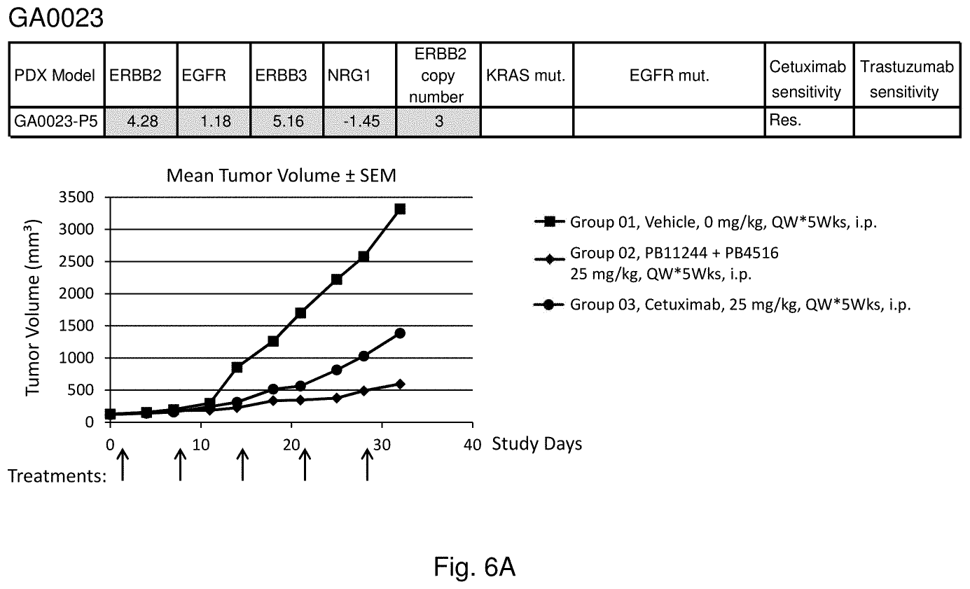

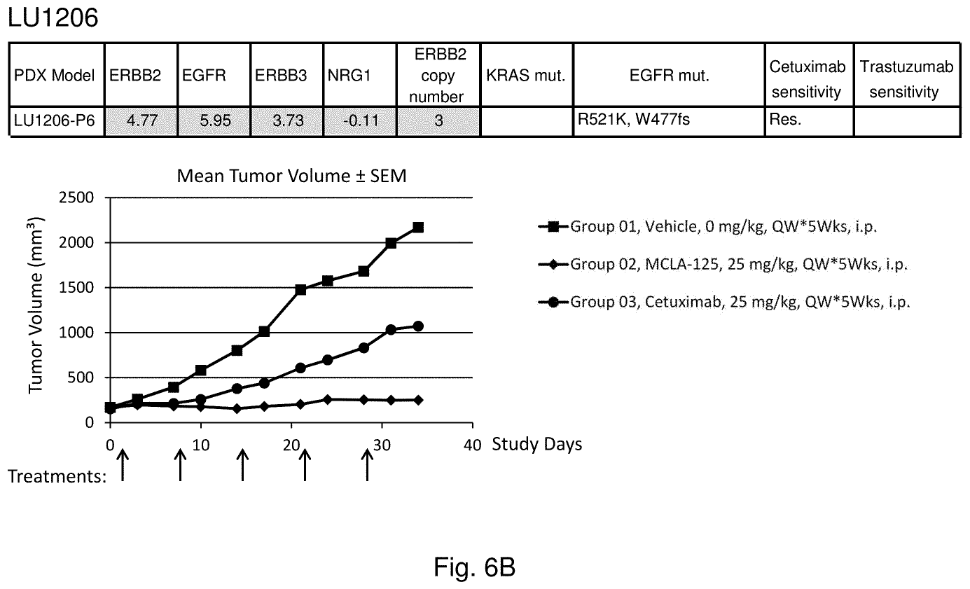

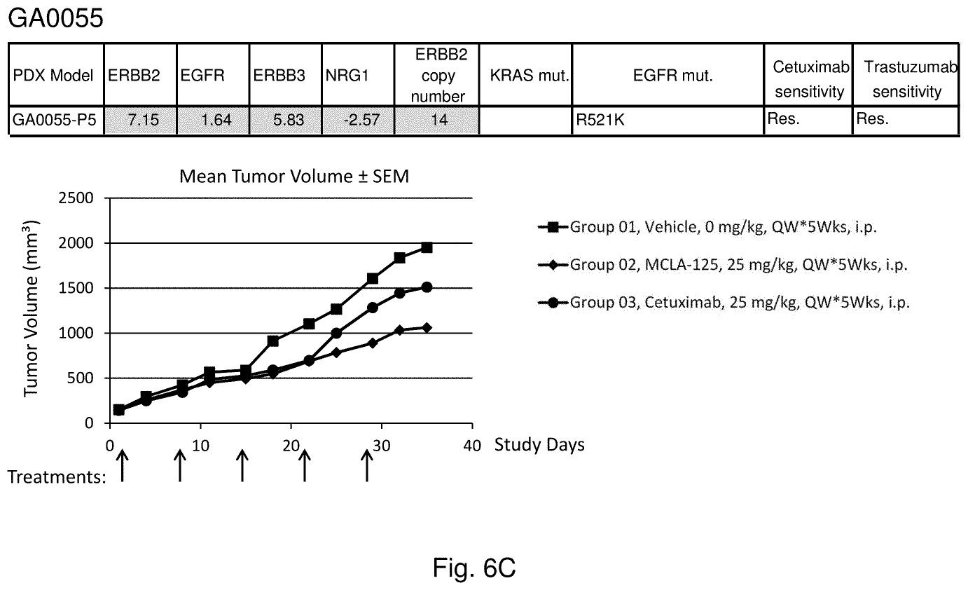

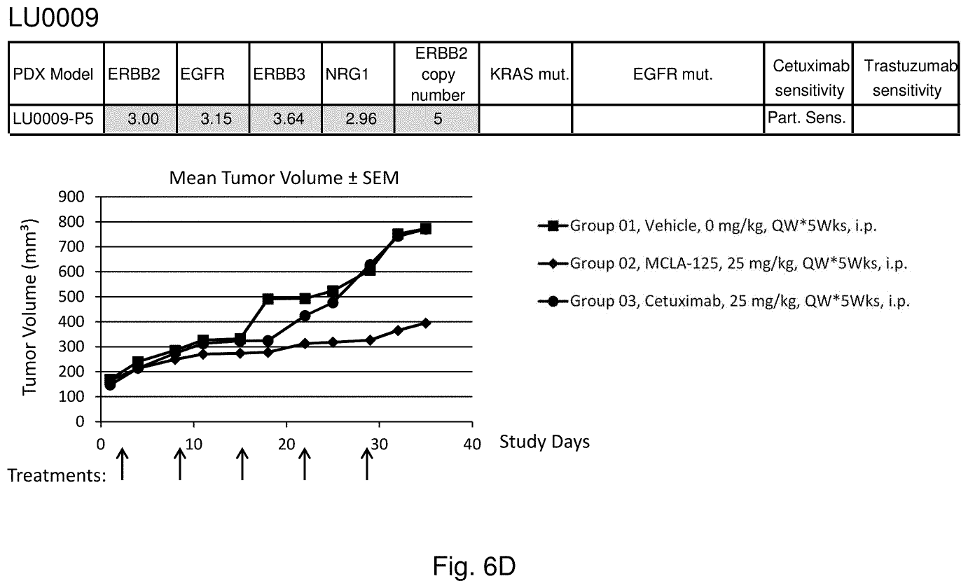

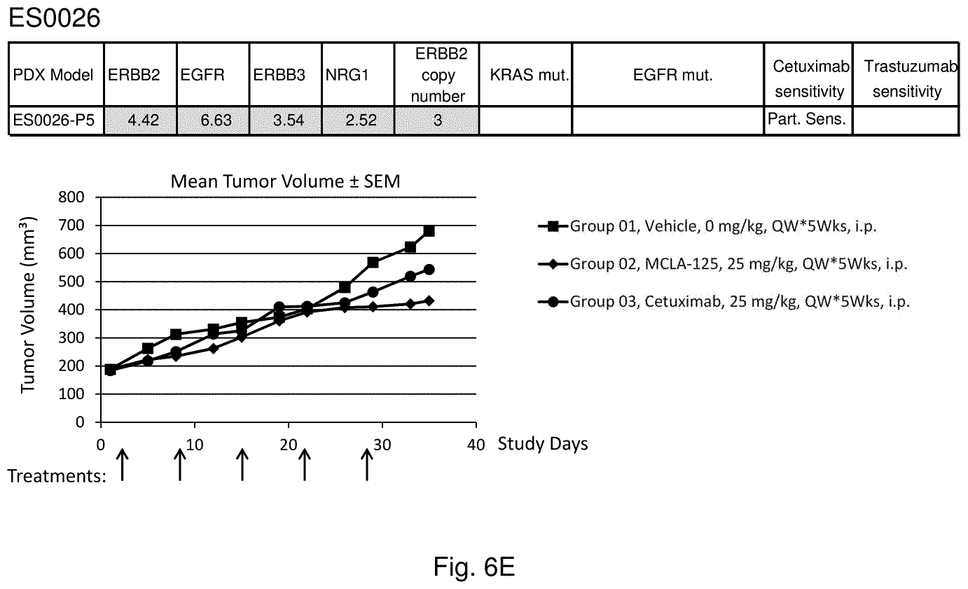

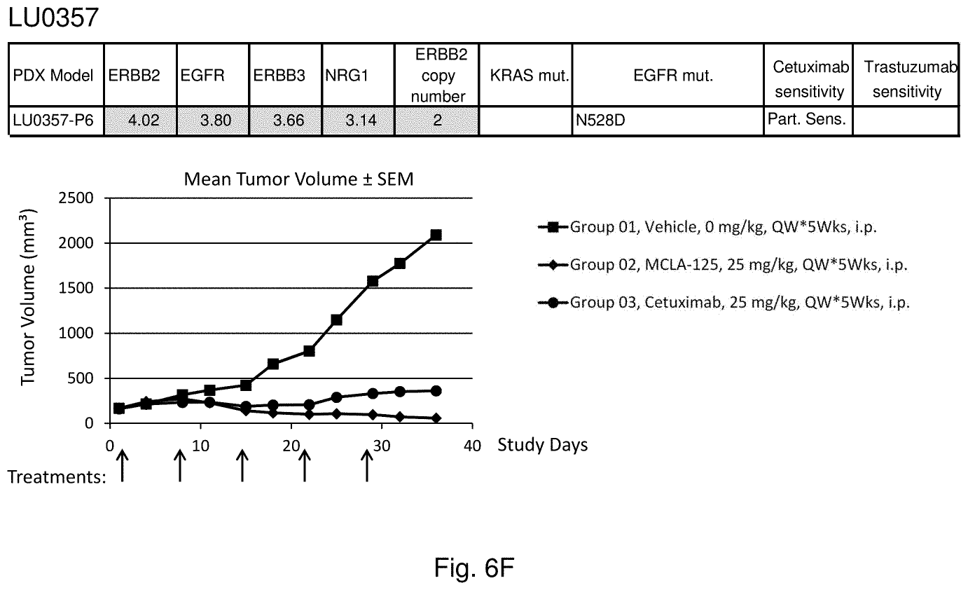

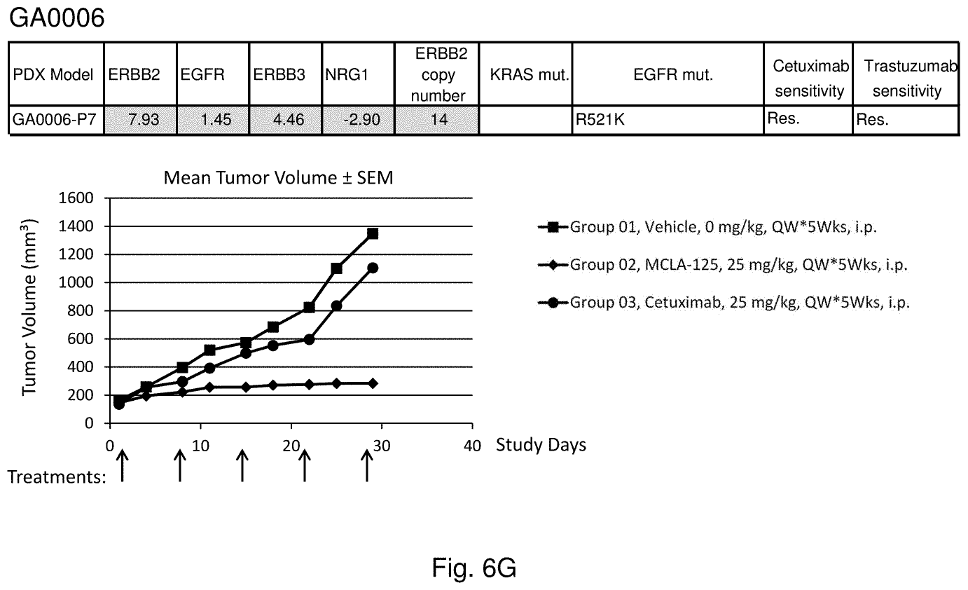

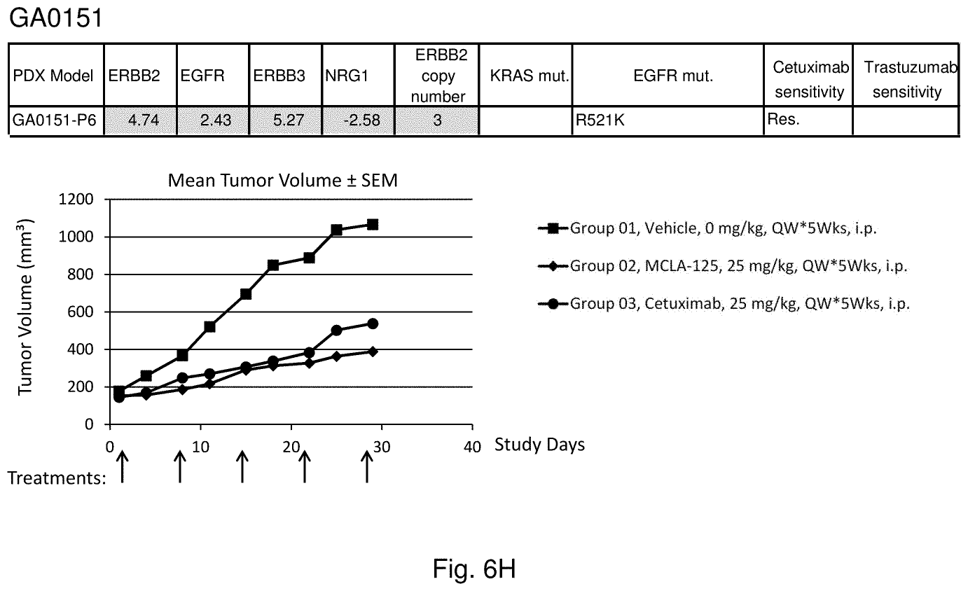

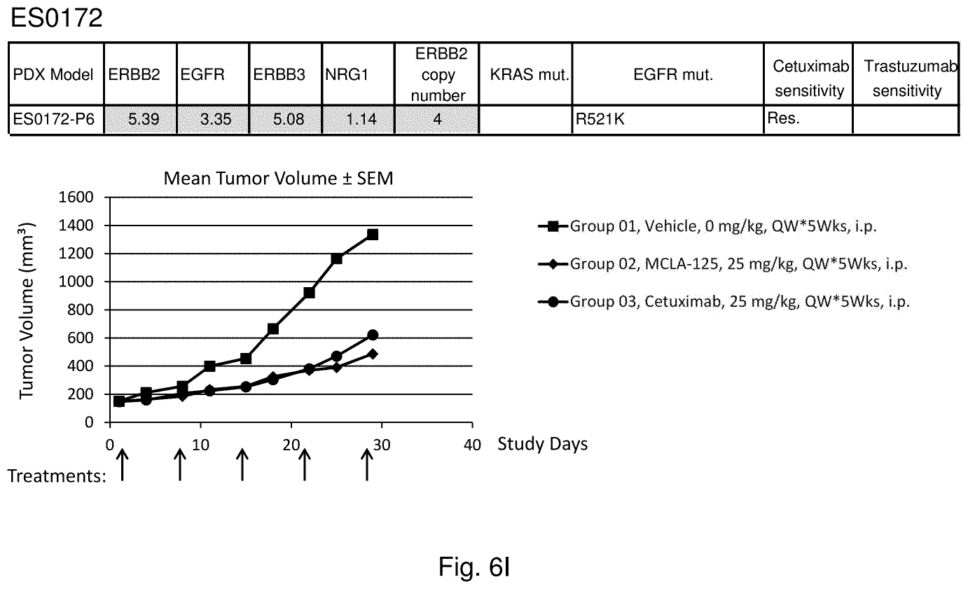

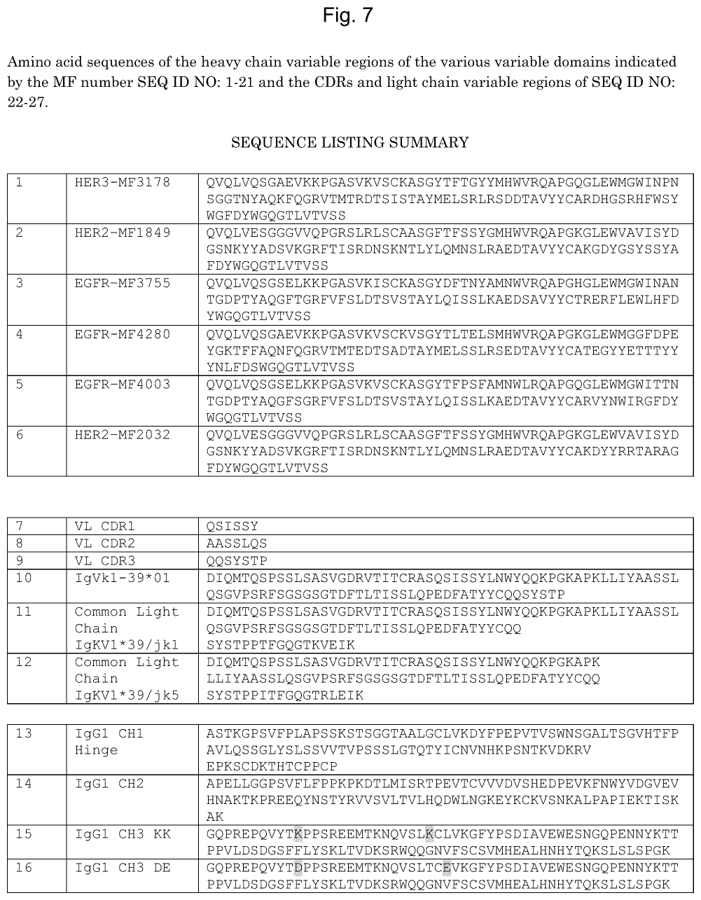

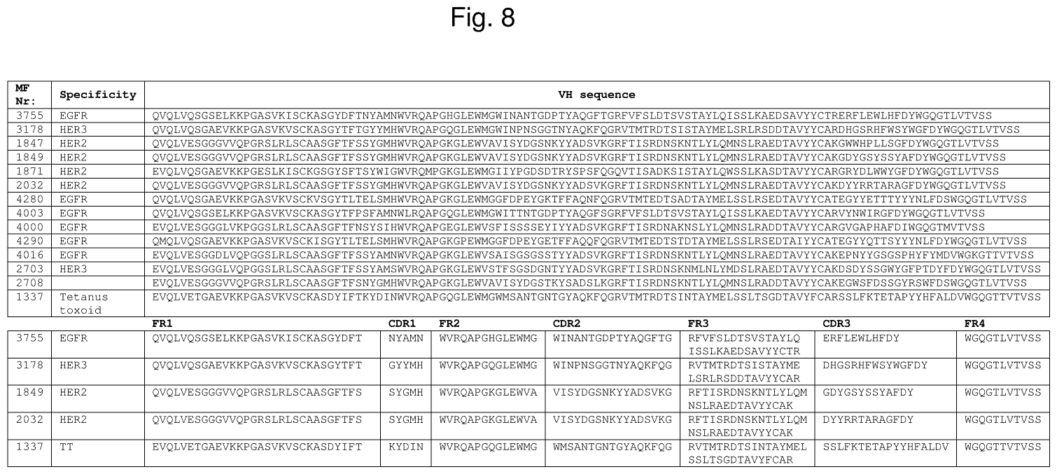

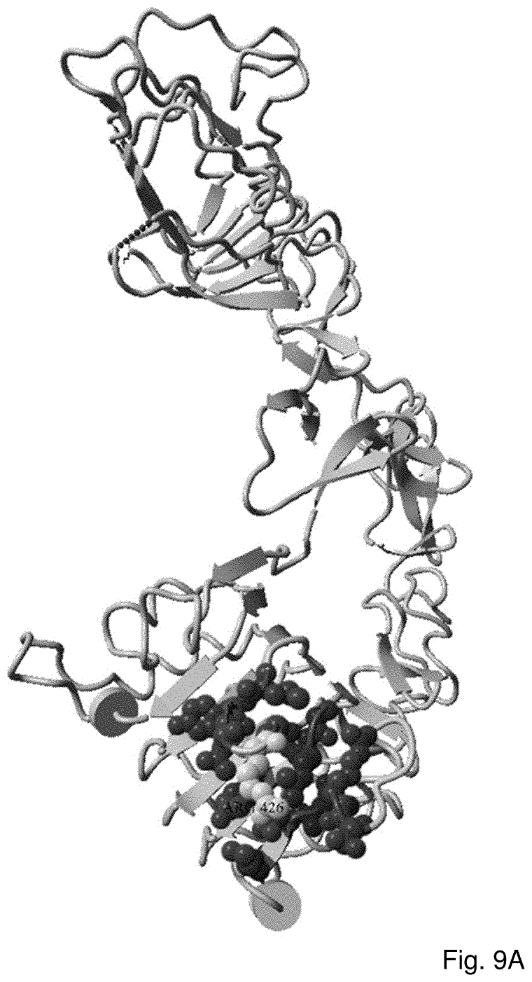

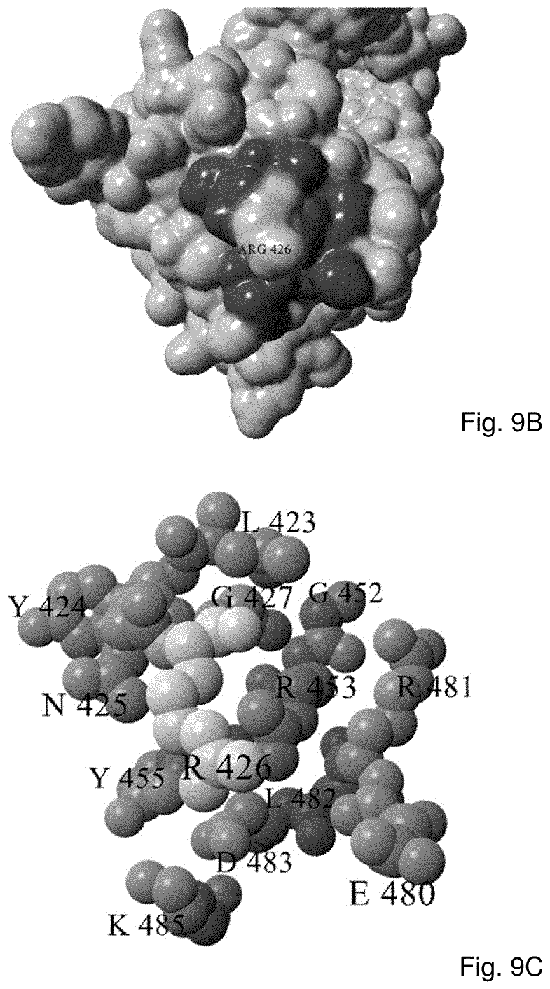

A schematic representation of embodiments wherein the composition comprises two bispecific antibodies that share a common arm. The figure depicts antibodies with heavy chains (1) and light chains (4). The four heavy chains have three different variable regions (5, 6 and 7). The heavy chain that has the shared variable region (5) has one part (3) of a heterodimerization domain. The heavy chains with variable regions (6) and (7) have the compatible part of the heterodimerization domain (2). Preferred pairing of heterodimerization regions (2) and (3) can direct formation of bispecific antibodies.

FIG. 2

Inhibitory effect of two Oligoclonics® on the proliferation of growth factor dependent cell lines BxPC-3-luc2 (Perkin Elmer 125058) and N87 cells (NCI-87 cell (ATCC® CRL-5822™).

Two Oligoclonics® were tested for effect on BxPC-3-luc2 (left hand panel) and N87 (right hand panel) cell proliferation. The result of the panel screening was compared with a combination of two monospecific antibodies (the EGFR binding antibody cetuximab and the HER3 monospecific antibody PG3178), or with the EGFR×HER3 binding bispecific antibody PB4522. The cells were grown in the presence of saturating amounts of HRG and EGF. The level of cell growth of the respective cells with HRG and EGF and without antibodies (basal w/ ligand) is indicated as well as the basal level without HRG and EGF and without antibodies (w/o ligand).

The monospecific antibody PG3178 has an IgG1 constant region and two variable domains with the heavy chain variable region of MF3178 of FIG. 7 or FIG. 8 and the common light chain variable region of sequence 11 of FIG. 7). The bispecific antibody PB4522 has an IgG1 constant region and two variable domains. The HER3 variable domain has the heavy chain variable region of MF3178 of FIG. 7 or FIG. 8. The EGFR variable domain has the heavy chain variable region of MF4280 of FIG. 7 or FIG. 8. The light chain variable region in both antibodies is the same and has the amino acid sequence of the common light chain variable region of sequence 11 of FIG. 7).

FIG. 3

ADCC activity of a panel of Oligoclonics®. The ADCC activity of a panel of Oligoclonics® was tested using N87 and a CD16/NFAT reporter assay. The bispecific antibodies have an IgG1 constant region and two variable domains. The amino acid sequence of the heavy chain variable regions of the variable domains is indicated in FIG. 7 or FIG. 8. The light chain variable region in the antibodies is the same and has the amino acid sequence of the common light chain variable region of sequence 11 of FIG. 7.

FIG. 4

The numbering and specificity of various Oligoclonics® and the ADCC activity thereof. “-” indicates that no activity was observed.

Each row represents an Oligoclonics® comprising two bispecific antibodies. The internal code of the bispecific antibodies is indicated in the columns Bispecific 1 and 2. The heavy chain variable region of the HER2, HER3 and EGFR binding domains is indicated in the columns marked MF A, MF B and MF C. MF numbers 3178 and 2703 form a HER3 binding variable domain in combination with the common light chain. MF numbers 4280, 3755, 4003, 4016 form an EGFR binding variable domain in combination with the common light chain and MF numbers 1871, 1847, 1849 and 2032 form a HER2 binding variable domain in combination with the common light chain. The bispecific antibodies have an IgG1 constant region and two variable domains. The amino acid sequence of the heavy chain variable regions of the variable domains is indicated in FIG. 7 or FIG. 8. The light chain variable region in the antibodies is the same and has the amino acid sequence of the common light chain variable region of sequence 11 of FIG. 7.

FIG. 5

In vivo testing of Oligoclonics®. BxPC-3-luc2 or N87 cells were injected into the xenograft model on day 0. The Oligoclonics® comprising bispecific antibodies PB4516×PB6892 (see FIG. 4) or controls were injected on days 1, 7, 14, 21 and 28. Antibodies were injected intraperitoneally at a dose of 25 mg/kg. Depicted are the results for the Oligoclonics® (PB4516 and PB6892). Vehicle and cetuximab served as controls.

FIG. 6

In vivo testing of Oligoclonics® comprising bispecific antibodies PB4516 and PB6892 in various PDX models.

PDX models were injected on day 0 and treatments with antibody or controls were clone on days 1, 7, 14, 21 and 28. Antibodies were injected intraperitoneally at a dose of 25 mg/kg.

FIG. 7

Amino acid sequences of the heavy chain variable regions of the various variable domains indicated by the MF number, see sequence numbers 1-6 and the CDRs and light chain variable regions see sequence numbers 7-12.

FIG. 8

Amino acid sequences of the various MFs referred to herein. FR1-4 refers to framework regions 1-4. CDR1-3 refers to complementarity-determining regions 1-3. TT is tetanus toxoid.

FIG. 9

a) HER3 crystal structure (PDB #4P59) showing residue Arg 426 in gray spheres and residues within an 11.2 Å radius from Arg 426 in black spheres, b) residue Arg 426 and distant residues shown in gray within a 11.2 Å radius from Arg 426 shown in black; c) Residues in region Arg 426 in light gray and surrounding residues (all labeled) in dark gray. Figures and analyses were made with Yasara (www.yasara.org).

### EXAMPLES

Cell Lines

Hek293 cells, NCI-87 cells (ATCC® CRL-5822™, BxPC-3 (ATCC CRL-1687), BxPC-3-luc2, and CHO-K1 were maintained in growth medium supplemented with 10% heat inactivated fetal bovine serum (FBS)

Generation of Bispecific Antibodies

Bispecific antibodies were generated using above described DEKK CH3 technology for efficient hetero-dimerization and formation of a bispecific antibody. The CH3 technology uses charge-based point mutations in the CH3 region to allow efficient pairing of two different heavy chain molecules as previously described (WO 2013/157954 A1).

A VH gene was cloned in one of two different backbone IgG1 vectors. Depending on the binding partner the VH was cloned in an IgG1 backbone comprising the CH3 variant with heterodimerization variant “DE” or in the IgG1 backbone comprising the complementary CH3 heterodimerization variant “KK”. In case of bi- or multispecific antibodies wherein two or more antibodies share a heavy chain. The shared chain preferably has the CH3 heterodimerization variant “DE” (also referred to as the DE-heavy chain) and the two or more unique heavy chains have the CH3 heterodimerization variant “KK” (also referred to as the KK-heavy chains).

Adherent Hek293 cells were cultivated in 6-well plates to a confluency of 80%. The cells were transiently transfected with the DNA-FUGENE mixtures and further cultivated. Seven days after transfection, supernatant was harvested and medium was refreshed. Fourteen days after transfection supernatants were combined and filtrated through 0.22 μM. The sterile supernatant was stored at 4° C. Suspension adapted Hek293 cells were cultivated in T125 flasks at a shaker plateau until a density of 3.0×10e6 cells/ml. Cells were seeded at a density of 0.3-0.5×10e6 viable cells/ml in each well of a 24-deep well plate. The cells were transiently transfected with the individual sterile DNA: PEI-MIX and further cultivated. Seven days after transfection, supernatant was harvested and filtrated through 0.22 μM. The sterile supernatant was stored at 4° C.

Generation of stable cell line pools that co-express two bispecific antibodies CHO cells were transfected with the three heavy chain constructs and a common light chain construct in a molar ratio of common light chain construct (cLC): EGFR heavy chain:HER2 heavy chain:HER3 heavy chain=2.5:2:1:1. Ten pools of stably transfected cells were obtained (A-J). ELISA analysis of anti-EGFR, anti-HER2 and anti-HER3 antibodies was performed on the day 3 and day 6 supernatants of the 10 pools. All 3 specificities could be detected in all pools.

Stable cell line clones that co-express two bispecific antibodies were generated by plating the pools in semi-solid medium. The plated cells were allowed to grow for 7-10 days. Two rounds of single cell cloning were carried out by seeding and picking of single colonies. The Oligoclonics were generated from a single cell by fed-batch fermentation.

Determination of Antibody Titers

Cell supernatants were diluted at 1:4 and 1:50 in PBS, based on the total IgG concentrations. Single-antigen ELISAs were first performed to detect the presence of all three species of antibodies. The following antigens were used at 2.5 μg/ml dilution, to coat the wells of an ELISA plate; recombinant-human EGFR-ECD Fc (R&D Systems, 344-ER), recombinant human ErbB2-ECD Fc (R&D Systems, 1129-ER) and recombinant human ErbB3-ECD Fc (R&D Systems, 348-ER).

Two sandwich ELISAs were then developed to detect and quantify the two bispecifics molecules allowing estimation of the ratio between the two bispecifics. For detection of EGFR×HER2 bispecific, EGFR-Fc (R&D Systems, 344-ER) antigen was coated on the wells and detected with ErbB2-Fc (R&D Systems, 1129-ER). For detection of EGFR×HER3 bispecific, EGFR-Fc antigen was coated on the wells and detected with ErbB3-Fc (R&D Systems, 348-RB).

IgG Purification.

Purification of IgG was performed using affinity chromatography. Purifications were performed under sterile conditions using vacuum filtration. First the pH of the medium was adjusted to pH 8.0 and subsequently the productions were incubated with protein A Sepharose CL-4B beads (50% v/v) (Pierce) for 2 H at 25° C. on a shaking platform at 600 rpm. Next the beads were harvested by vacuum filtration. Beads were washed twice with PBS pH 7.4. IgG was eluted at pH 3.0 with 0.1 M citrate buffer and the IgG fraction was immediately neutralized by Tris pH 8.0. Buffer exchange was performed by centrifugation using Ultracel (Millipore). The samples ended up in a final buffer of PBS pH 7.4.

Cation-Exchange Chromatography (CIEX)

CEX-HPLC chromatography was done using TSKgel SP-STAT (7 μm particle size, 4.6 mM I.D.×10 cm L, Tosoh 21964) series of ion exchange columns. The columns are packed with non-porous resin particles for speed and high resolution analysis, as well as isolation, of biomolecules. The particles in TSKgel STAT columns contain an open access network of multi-layered ion-exchange groups for loading capacity, while the relatively large particle size makes these columns suitable for HPLC and FPLC systems.

The TSKgel SP-STAT (7 μm particle size, 4.6 mM I.D×10 cm L, Tosoh 21964) is equilibrated using Buffer A (Sodium Phosphate buffer, 25 mM, pH 6.0), after which antibodies are displaced from the column by increasing salt concentration and running a gradient of Buffer B (25 mM Sodium Phosphate, 1 mM NaCl, pH 6.0). Flow rate was set at 0.5 mL/min. The injection sample mass for all test samples and controls (in PBS) was 10 μg and injection volumes 10-100 μl. The chromatograms are analyzed for peak patterns, retention times and peak areas for the major peaks observed based on the 220 nm results.

BxPC-3-luc2 and N87 Growth Inhibition Assay

The antibody compositions were tested at a concentration range of total antibody. The antibodies were pooled two by two on the basis of equal amounts of weight per weight. The HRG and EGF were added to the culture at 0.1 ng/ml of EGF and 10 ng/ml of HRG for BxPC3-luc2 cells, or 0.1 ng/ml of EGF and 1 ng/ml of HRG for N87 cells. Basal w/ ligand was a control without antibody but with the respective growth factors. Basal w/o ligand was a control without the indicated growth factors and without antibody.

Antibodies were diluted in chemically defined starvation medium (CDS: RPM11640 medium, containing 80 U penicillin and 80 μg of streptomycin per ml, 0.05% (w/v) BSA and 10 μg/ml holo-transferrin) and 50 μl of diluted antibody was added to the wells of a 96 wells black well clear bottom plate (Costar). Ligands were added (50 μl per well of a stock solution containing 40 ng/ml or 4 ng/ml HRG and 400 ng/ml of EGF, diluted in CDS: R&D systems, cat. nr. 396-HB and 236-EG). Plates were left for an hour at rt before being put in a container inside a 37° C. cell culture incubator for three days (N87 cells) or four days (BxPC-3-luc2 cells). On the fourth day. Alamar blue (Invitrogen, #DAL1100) was added (20 μl per well) and the fluorescence was measured after 6 hours (N87 cells) or four hours (BxPC-3-luc2 cells) of incubation (at 37° C.) with Alamar blue using 560 nm excitation and 590 nm readout on a Biotek Synergy 2 Multi-mode microplate reader. Fluorescence values were normalised to uninhibited growth (no antibody, but both ligands added).

ADCC Activity of the Various Oligoclonics®

The ADCC Reporter Bioassay (Promega) was used. Two different cell lines were tested; the EGFR expressing pancreatic cancer cell line BxPC3 and the gastric carcinoma N87 cell line.

The bioassay uses engineered Jurkat cells stably expressing either the FcγRIIIa receptor V158 (high affinity) variant, and an NFAT response element driving expression of firefly luciferase which is a measure for FcγR activation. The assay has been validated by comparing data obtained with this ADCC Reporter Bioassay to the classical 51Cr release assay and both assays yield similar results. The ADCC assays were performed using the Promega ADCC Bioassay kit using 384 white well plates. In this experimental setup BxPC3 cells and N87 cells were plated at a density of 1000 cells/well in 30 μl assay medium (RPM with 4% low IgG serum) 20-24 H before the bioassay. The next day, the culture medium was removed. Next, a serial dilution of the Oligoclonics® and a comparator antibody cetuximab were prepared in duplicates. 10 μl of these antibody dilutions were added to the wells. Control wells without antibody were also included (basal). From the starting concentrations of the antibodies 5-fold serial dilutions were generated to provide dose-response curves. Finally, 5 μl of ADCC Bioassay effector cells (15000 cells/well, V158) was added. The cells were incubated for Gil at 37° C. Next, 15 μl BIO-Glo luciferase substrate was added and 5 minutes later luminescence was detected in a plate reader. The obtained data are shown in FIG. 3. Cetuximab showed ADCC activity towards the BxPC3 and N87 cells. Various oligoclonic antibodies also showed ADCC activity on BxPC3 and/or N87 cells.

Testing the Oligoclonics® Comprising Bispecific Antibodies PB-1516 and PB6892 for its Effect on the Growth of BxPC-3-luc2 Tumours (Orthotopically Implanted) and N87 Tumours (Gastric Cells Implanted in the Flank).

CB17 SCID female mice, 8-10 weeks old at the beginning of the study were engrafted orthotopically in the pancreas with 1×10e6 BxPC-3-luc2 tumor cells in 20 μl. Mice are anesthetized and laid on the right side to expose the left side and a 0.5 cm incision is made on the left flank region. The pancreas and spleen were exteriorized and 1×10e6 tumor cells in 20 μl were injected into the sub-capsulary space of the pancreas tail. One week after implantation, bioluminescence (BLI) data were generated. For BLI imaging (once or twice weekly) left side view, all mice received 15 minutes prior to the imaging all of the mice receive i.p. injections of 150 mg/kg Luciferin (D-Luciferin-EF Potassium Salt, Cat. #E6552, Promega).

Outlier animals—based on BLI/tumor volume—were removed and the mice were randomly distributed into groups of 7 mice each. On experimental day 8, the treatment was started.

The animals in the antibody treatment group were dosed weekly for 4 consecutive weeks (days 0, 7, 14 and 21) with 0.3 mg/kg of antibody. At day 0 of the treatment the animals receive twice the loading dose, i.e. 0.6 mg/kg of antibody. The final imaging was carried out at day 35 or day 40. Vehicle only and cetuximab treated groups served as controls.

Cetuximab and the oligoclonic significantly decrease BxPC-3 tumour outgrowth in the model (p<0.05) (FIG. 5). Tumor outgrowth with the Oligoclonics® PB4516 and PB6892 was notably less than with cetuximab. Cetuximab did not significant reduce the outgrowth of N87 calls. The Oligoclonics® significantly decreased N87 tumour outgrowth in the model (p<0.05) (FIG. 5).

N87 Tumour:

CB17 SCID female mice, 8-12 weeks old at the beginning of the study, were inoculated with 1×10e7 N87 tumor cells in 50% Matrigel sc in flank. Cell injection volume was 0.2 mL/mouse. Treatment was started when tumors reached an average size of 150-200 mm3. Antibodies were administered once a week for 4 weeks by intraperitoneal injections at 25 mg/kg of mice. Body weight was measured once a week after tumor cell injection and biweekly after the start of the treatment to the end. Tumor growth was monitored by caliper measurements biweekly. The end point of the experiment was a tumor volume of 800 mm3 or 60 days, whichever came first.

Activity of the Oligoclonics® PB11244 and PB4516 in Various PDX Models.

The activity of the Oligoclonics® comprising bispecific antibodies PB11244 and PB4516 was assessed in a suite of PDX models. Testing candidate therapeutics in large number of cancer models facilitates predictions of clinical efficacy and can identify factors for patient-selection strategies.

The bispecific antibodies PB4516 and PB11244 have an IgG1 constant region and two variable domains.

The HER3 variable domain of PB4516 has the heavy chain variable region of MF3178 of FIG. 7 or FIG. 8. The EGFR variable domain has the heavy chain variable region of MF3755 of FIG. 7 or FIG. 8.

The HER2 variable domain of PB11244 has the heavy chain variable region of MF2032 of FIG. 7 or FIG. 8. The EGFR variable domain has the heavy chain variable region of MF3755 of FIG. 7 or FIG. 8.

The light chain variable region in both antibodies was the same and had the amino acid sequence of the common light chain variable region of SEQ ID NO: 11 of FIG. 7)

A selection of several gastric, oesophageal and non-small cell lung cancer PDX models was made (FIG. 6).

The Oligoclonics® comprising bispecific antibodies PB4516 and PB11244 were produced and purified. The antibodies were mixed in a 1:1 ratio. The Oligoclonics® was tested in the models and compared to cetuximab and vehicle (PBS).

The PDX models were first expanded subcutaneously (s.c.) in donor BALB/c nude mice. Tumors were extracted, cut into small pieces (2-3 mm in diameter) and implanted s.c. in new acceptor BALB/c nude mice. Recipients for tumours were female BALB/c nude mice that are 6-8 weeks of age. Tumor growth was followed by Caliper measurement until tumors reached an average size of 100-200 mm3. At this stage, noted as day 1, animals were randomized into 3 groups for each model. Treatments were initiated on the same day and included:
PB4516×PB689225 mg/kg, 5 weekly doses, intraperitoneal injection
Cetuximab 25 mg/kg, 5 weekly doses, intraperitoneal injection
Vehicle (PBS), 5 weekly doses, intraperitoneal injection

It can be seen that the Oligoclonics® significantly reduced the outgrowth of the tumour cells in the model. The reduction of outgrowth was equal or better than cetuximab.

TABLE 1
List of residues within 11.2 Å radius of Arg 426 in HER3:

| | |
|---|---|
| Leu 423 | L423 |
| Tyr 424 | Y424 |
| Asn 425 | N425 |
| Gly 427 | G427 |
| Gly 452 | G452 |
| Arg 453 | R453 |
| Tyr 455 | Y455 |
| Glu 480 | E480 |
| Arg 481 | R481 |
| Leu 482 | L482 |
| Asp 483 | D483 |
| Lys 485 | K485 |

1. A composition comprising two or more binding moieties,
wherein each of said binding moieties comprises a variable domain that binds to an extracellular part of EGFR; and
wherein a first of said binding moieties comprises a variable domain that binds to an extracellular part of HER2 and a second of said binding moieties comprises a variable domain that binds to an extracellular part of HER3.

2. The composition of claim 1, wherein at least one and preferably at least two of the two or more binding moieties is an antibody.

3. The composition of claim 1 or claim 2, wherein at least one and preferably at least two of the two or more binding moieties is an IgG.

4. The composition of claim 2 or claim 3, wherein the CH3-regions of the heavy chains of a first and/or a second antibody are engineered to facilitate heterodimerization of a heavy chain with an EGFR binding variable domain with a heavy chain with an HER2 binding variable domain and/or an EGFR binding variable domain with a heavy chain with an HER3 binding variable domain.

5. The composition of any one of claims 2 to 4, wherein at least one, and preferably at least two of the two or more antibodies is a bispecific antibody.

6. The composition of any one of claims 2 to 5, wherein the variable domains that bind to an extracellular part of EGFR of the first and second antibody comprise substantially the same heavy chain variable region.

7. The composition of any one of claims 2 to 6, wherein the variable domain that binds to an extracellular part of EGFR binds domain I or domain III of EGFR, preferably domain III.

8. The composition of any one of claims 2 to 7, wherein the variable domain that binds to an extracellular part of HER2 binds domain I or domain IV of HER2, preferably domain IV.

9. The composition of any one of claims 2 to 8, wherein the variable domain that binds to an extracellular part of HER3 binds domain III of HER3.

10. The composition of any one of claims 6 to 9, wherein the variable domain that binds to an extracellular part of EGFR binds domain I or domain III of EGFR, preferably domain III; wherein the variable domain that binds to an extracellular part of HER2 binds domain I or domain IV of HER2, preferably domain IV; and wherein the variable domain that binds to an extracellular part of HER3 binds domain III of HER3.

11. The composition of claim 9 or claim 10, wherein the variable domain that binds to an extracellular part of HER3 binds at least to R426 of domain III of HER3

12. The composition of any one of claims 9 to 11, wherein the affinity (KD) of the variable domain that binds to an extracellular part of HER3, for binding to an HER3 positive SK-BR-3 cell (ATCC® HTB-30™), is lower than or equal to 2.0 nM, preferably from 2.0 to 0.1 nM.

13. The composition of any one of claims 1 to 12, wherein the binding of the variable domain that binds EGFR to EGFR blocks the binding of EGF to EGFR and/or wherein the binding of the variable domain that binds HER3 to HER3 blocks the binding of neuregulin 1 (NRG) to HER3.

14. The composition of any one of claims 1-13, wherein the variable domain that binds to an extracellular part of EGFR comprises a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs.

15. The composition of any one of claims 1-14, wherein the variable domain that binds to an extracellular part of HER2 comprises a heavy chain variable region comprising a CDR1 sequence SYGMH, a CDR2 sequence VISYDGSNKYYADSVKG and a CDR3 sequence DYYRRTARAGFDY or a variant thereof comprising a substitution, deletion and/or insertion of 1, 2, or 3 amino acids in the CDRs.

16. The composition of any one of claims 1-15, wherein the variable domain that binds to an extracellular part of HER3 comprises a heavy chain variable region comprising a CDR1 sequence GYYMH, a CDR2 sequence WINPNSGGTNYAQKFQG and a CDR3 sequence DHGSRHFWSYWGFDY or a variant thereof comprising a substitution, deletion and/or insertion of 1, 2, or 3 amino acids in the CDRs.

17. The composition of any one of claims 1-16
wherein the variable domain that binds to an extracellular part of EGFR comprises a heavy chain variable region comprising a CDR1 sequence NYAMN, a CDR2 sequence WINANTGDPTYAQGFTG and a CDR3 sequence ERFLEWLHFDY or a variant thereof comprising a substitution, deletion and/or insertion of 1, 2, or 3 amino acids in the CDRs;
wherein the variable domain that binds to an extracellular part of HER2 comprises a heavy chain variable region comprising a CDR1 sequence SYGMH, a CDR2 sequence VISYDGSNKYYADSVKG and a CDR3 sequence DYYRRTARAGFDY or a variant thereof comprising a substitution, deletion and/or insertion of 1, 2, or 3 amino acids in the CDRs; and
wherein the variable domain that binds to an extracellular part of HER3 comprises a heavy chain variable region comprising a CDR1 sequence GYYMH, a CDR2 sequence WINPNSGGTNYAQKFQG and a CDR3 sequence DHGSRHFWSYWGFDY or a variant thereof comprising a substitution, deletion and/or insertion of 1, 2, or 3 amino acids in the CDRs.

18. A composition of any one of claims 1 to 17 for use in treatment.

19. A composition according to claim 18 for use in the treatment of cancer, preferably gastric cancer, lung cancer or esophagus cancer.

20. A pharmaceutical composition comprising a composition according to any one of claims 1 to 17.

21. Two or more binding moieties that each comprise a variable domain that binds to an extracellular part of EGFR; wherein a first of said binding moieties comprises a variable domain that binds to an extracellular part of HER2 and a second of said binding moieties comprises a variable domain that binds to an extracellular part of HER3 for use in the treatment of cancer, preferably gastric cancer, lung cancer or esophagus cancer.

22. A product containing two or more binding moieties, wherein each of said binding moieties comprises a variable domain that binds to an extracellular part of EGFR; and wherein a first of said binding moieties comprises a variable domain that binds to an extracellular part of HER2 and a second of said binding moieties comprises a variable domain that binds to an extracellular part of HER3 as a combined preparation for simultaneous, separate or sequential use in treating cancer preferably gastric cancer, lung cancer or esophagus cancer.

23. The composition, pharmaceutical composition, binding moieties or product for use of any one of claims 18-22, wherein the cancer comprises cells with an EGFR-mutation that renders the cell resistant to treatment with a tyrosine kinase inhibitor (TKI).

24. The composition, pharmaceutical composition, binding moieties or product for use of any one of claims 18-23, wherein the cancer comprises cells with an EGFR R521K polymorphism.

25. The composition, binding moieties or product for use of any one of claims 1-24, wherein the cancer is gastric cancer.

26. A method for the treatment of a subject that has cancer or is at risk of recurrence or relapse of cancer, the method comprising administering to a subject in need thereof a therapeutically effective amount of two or more binding moieties, wherein each of said binding moieties comprises a variable domain that binds to an extracellular part of EGFR; and wherein a first of said binding moieties comprises a variable domain that binds to an extracellular part of HER2 and a second of said binding moieties comprises a variable domain that binds to an extracellular part of HER3.

27. A method for producing a composition according to any one of claims 1-19, which method comprises:
providing a cell comprising
a nucleic acid that encodes a polypeptide comprising a heavy chain that is capable of pairing with a common light chain to form a variable domain that binds to an extracellular part of EGFR;
a nucleic acid that encodes a polypeptide comprising a heavy chain that is capable of pairing with said common light chain forms a variable domain that binds to an extracellular part of HER2;
a nucleic acid that encodes a polypeptide comprising a heavy chain that is capable of pairing with said common light chain forms a variable domain that binds to an extracellular part of HER3; and
a nucleic acid that encodes a polypeptide comprising said common light chain;
wherein, optionally two or more of said nucleic acids may be physically linked, and wherein each of said nucleic acids further comprises an expression control sequence to allow expression of the encoded heavy and light chains in said cell and;
culturing said cell to allow expression of said heavy and light chains; and, optionally,
recovering said two or more binding moieties.

28. The method of claim 27 comprising providing a plurality of cells with said nucleic acids and selecting from said collection a cell with a desired ratio of expression of the heavy and light chains.

29. The method of claim 27 or claim 28, wherein said two or more binding moieties are antibodies, preferably bispecific antibodies.

30. The method of any one of claims 27 to 29, wherein the cells produce essentially equimolar amounts of the two or more binding moieties.

31. The method of any one of claims 27-30, wherein the cells produce more of a first binding moiety than of a second of said two or more binding moieties.

32. A cell comprising
a nucleic acid that encodes a polypeptide comprising a heavy chain that together with a common light chain forms a variable domain that binds to an extracellular part of EGFR;
a nucleic acid that encodes a polypeptide comprising a heavy chain that together with said common light chain forms a variable domain that binds to an extracellular part of HER2;
a nucleic acid that encodes a polypeptide comprising a heavy chain that together with said common light chain forms a variable domain that binds to an extracellular part of HER3; and
a nucleic acid that encodes a polypeptide comprising said common light chain;
wherein two or more of said nucleic acids may be physically linked or not and wherein each of said nucleic acids further comprises an expression control sequence to allow expression of the encoded heavy and light chains in said cell.

33. A container comprising nucleic acid comprising
a nucleic acid that encodes a polypeptide comprising a heavy chain that is capable of pairing with a common light chain forms a variable domain that binds to an extracellular part of EGFR;
a nucleic acid that encodes a polypeptide comprising a heavy chain that is capable of pairing with a common light chain forms a variable domain that binds to an extracellular part of HER2;
a nucleic acid that encodes a polypeptide comprising a heavy chain that is capable of pairing with a common light chain forms a variable domain that binds to an extracellular part of HER3; and
a nucleic acid that encodes a polypeptide comprising said common light chain;
wherein, optionally two or more of said nucleic acids may be physically linked, and wherein each of said nucleic further comprises an expression control sequence to allow expression of the encoded heavy and light chains in a cell.
